(19) United States
(12) Patent Application Publication
(10) Pub. No.: US 2006/0073159 A1
(43) Pub. Date: Apr. 6, 2006

(54) Human anti-cancer immunotherapy

(75) Inventors: Robert H. Vonderheide, Merion Station, PA (US); Gregory Beatty, Kennett Square, PA (US); Christina Coughlin, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Trustees of the University of Pennsylvania

(21) Appl. No.: 11/137,253

(22) Filed: May 25, 2005

Related U.S. Application Data
(60) Provisional application No. 60/574,017, filed on May 25, 2004.

(51) Int. Cl. A61K 39/00; A61K 48/00

(52) U.S. Cl. 424/185.1; 514/44

(57) ABSTRACT

The present invention encompasses compositions and methods for activating, stimulating and isolating antigen-specific T cells. The present invention also relates to compositions of antigen-specific T cells and methods of their use in the treatment and prevention of cancer, infectious diseases, autoimmune diseases, immune disfunction related to aging, or any other disease state where antigen-specific T cells are desired for treatment.

### CROSS-REFERENCE TO RELATED APPLICATIONS

This application claims priority under 35 U.S.C. § 119(e) to U.S. Provisional Patent Application No. 60/574,017, filed May 25, 2004, which is incorporated by reference herein in its entirety.

### BACKGROUND OF THE INVENTION

The ability of T cells to recognize an antigen is dependent on the association of the antigen with either major histocompatibility complex (MHC) I or MHC II proteins. For example, cytotoxic T cells respond to an antigen that is presented in association with MHC-I proteins. Thus, a cytotoxic T cell that should kill virus-infected cell will not kill that cell if the cell does not also express the appropriate MHC-I protein. Helper T cells recognize MHC-II proteins. Helper T cell activity depends, in general, on both the recognition of the antigen on antigen presenting cells and the presence on these cells of “self” MHC-II proteins. The requirement for recognition of an antigen in association with a self-MHC protein is called MHC restriction. MHC-I proteins are found on the surface of virtually all nucleated cells. MHC-II proteins are found on the surface of certain cells including macrophages, B cells, and dendritic cells of the spleen and Langerhans cells of the skin.

A crucial step in mounting an immune response in mammals, is the activation of CD4+ helper T-cells that recognize MHC-II restricted exogenous antigens. These antigens are captured and processed in the cellular endosomal pathway in antigen presenting cells, such as dendritic cells (DCs). In the endosome and lysosome, the antigen is processed into small antigenic peptides that are complexed onto the MHC-II in the Golgi compartment to form an antigen-MHC-II complex. This complex is expressed on the cell surface, which expression induces the activation of CD4+ T cells.

Other crucial events in the induction of an effective immune response in an mammal involve the activation of CD8+ T-cells and B cells. CD8+ cells are activated when the desired protein is routed through the cell in such a manner so as to be presented on the cell surface as a processed protein, which is complexed with MHC-I antigens. B cells can interact with the antigen via their surface immunoglobulins (IgM and IgD) without the need for MHC proteins. However, the activation of the CD4+ T-cells stimulates all arms of the immune system. Upon activation, CD4+ T-cells (helper T cells) produce interleukins. These interleukins help activate the other arms of the immune system. For example, helper T cells produce interleukin-4 (IL-4) and interleukin-5 (IL-5), which help B cells produce antibodies; interleukin-2 (IL-2), which activates CD4+ and CD8+ T-cells; and gamma interferon, which activates macrophages. Since helper T-cells that recognize MHC-II restricted antigens play a central role in the activation and clonal expansion of cytotoxic T-cells, macrophages, natural killer cells and B cells, the initial event of activating the helper T cells in response to an antigen is crucial for the induction of an effective immune response directed against that antigen.

In addition to the critical roles that T cells play in the immune response, antigen presenting cells (APCs) are equally important. An example of an APC is a dendritic cell (DC), which is a professional antigen-presenting cell having a key regulatory role in the maintenance of tolerance to self-antigens and in the activation of innate and adaptive immunity (Banchereau et al., 1998, Nature 392:245-52; Steinman et al., 2003, Annu. Rev. Immunol. 21:685-711). When DCs encounter pro-inflammatory stimuli such as microbial products, the maturation process of the cell is initiated by up-regulating cell surface expressed antigenic peptide-loaded MHC molecules and co-stimulatory molecules. Following maturation and homing to local lymph nodes, DCs establish contact with T cells by forming an immunological synapse, where the T cell receptor (TCR) and co-stimulatory molecules congregate in a central area surrounded by adhesion molecules (Dustin et al., 2000, Nat. Immunol. 1:23-9). Once activated, CD8+ T cells can autonomously proliferate for several generations and acquire cytotoxic function without further antigenic stimulation (Kaech et al., 2001, Nat. Immunol. 2:415-22; van Stipdonk et al., 2001, Nat. Immunol. 2:423-9). It has therefore been proposed that the level and duration of peptide-MHC complexes (signal 1) and co-stimulatory molecules (signal 2) provided by DCs are essential for determining the magnitude and fate of an antigen-specific T cell response (Lanzavecchia et al., 2001, Nat. Immunol. 2:487-92; Gett et al., 2003, Nat. Immunol. 4:355-60).

Experiments in animal models have demonstrated the potential of immune-based approaches for cancer therapy. Antibody and cytokine therapy have already been successfully developed and incorporated into standard treatment regimens for some human malignancies. The development of cellular immunotherapy with effector cells of defined specificity and function has proven more challenging.

In murine models, the adoptive transfer of CD8+ or CD4+ T cells specific for tumor associated or minor histocompatibility antigens (mHAgs) expressed by leukemic cells provides a potent antileukemic effect and converts the incomplete responses achieved with chemotherapy into cure (Pion et al., 1995, J. Clin. Invest. 95:1561-1568; Greenberg 1991, Adv. Immunol. 49:281-355; Fontaine et al., 2001, Nat. Med. 7:789-794). There exist evidence, much of which is derived from the results of allogeneic hematopoietic stem cell transplantation (HSCT), that human leukemias can also be recognized and eliminated by T cells. The immunologically mediated graft-vs-leukemia (GVL) effect that was predicted by animal model studies of allogeneic HSCT has been documented in clinical trials. Patients who receive an allogeneic transplant for advanced leukemia have a lower probability of leukemic relapse if they develop acute and/or chronic graft-vs-host disease (GVHD) as a complication of the transplant (Weiden et al., 1979, N. Engl. J Med. 300:1068-1073; Weiden et al., 1981, N. Engl. J. Med. 304:1529-1533). The risk of leukemic relapse is increased after syngeneic HSCT or T-cell depleted allogeneic HSCT, suggesting a critical role for donor T cells specific for allogeneic determinants in initiating or mediating the GVL effect. Although the GVL effect is most prominent in patients with GVHD, a reduction in relapse is also evident in patients without GVHD, thus demonstrating that clinical GVHD is not a prerequisite for GVL activity (Horowitz et al., 1990, Blood 75:555-562). The type of leukemia is also a factor in the GVL effect associated with allogeneic HSCT. The reduction in relapse attributed to donor T cells is greatest for chronic myeloid leukaemia (CML), intermediate for acute myeloid leukemia (AML), and lowest for acute lymphoblastic leukemia (ALL) (Horowitz et al., 1990, Blood 75:555-562). The importance of the GVL effect to a successful outcome after allogeneic HSCT is well established, but relapse and GVHD remain significant obstacles, especially for patients with advanced acute leukemia.

Most of the investigation into cellular immunotherapy of leukemia has been concentrated on identifying T-cell responses to candidate proteins expressed in leukemic cells. Two subsets of mature T cells express the αβT-cell receptor. CD3+ CD8+ cytotoxic T cells recognize short peptides of 8-11 amino acids derived from intracellular proteins and displayed on the surface of cells associated with class I MHC molecules. CD3+ CD4+ helper T cells recognize peptides derived from intracellular proteins or proteins that have been taken up by endocytosis and presented at the cell surface by class II MHC molecules. Both subsets of T cells have antileukemic activity in animal models.

Several classes of proteins expressed by leukemic cells have been identified to provide peptide epitopes that are recognized by CD8+ or CD4+ T cells. These include minor histocompatibility antigens (mHAgs) that are relevant as targets after allogeneic HSCT, and leukemia-specific or leukemia-associated proteins that may be targets in both transplant and nontransplant settings.

Leukemia-specific proteins that are expressed as a consequence of chromosome translocations or mutations in cellular genes represent one category of candidate antigens for T-cell immunotherapy. Examples of this class of proteins include the bcr/abl fusion protein resulting from the t9,22 translocation in CML, the PML/RAR α fusion protein resulting from the t15,17 translocation in acute promyelocytic leukemia, and the ETV6-AML1 fusion protein in childhood ALL (Bocchia et al., 1996, Blood 87:3587-3592; Yotnda et al., 1998, J. Clin. Invest. 102:455-462; Yasukawa et al., 2001, Blood 98:1498-1505). These proteins are attractive for immunotherapeutic approaches because they exhibit selective expression on tumor cells, which limits the potential for toxicity to normal tissues,and may contribute to the malignant phenotype, which makes it less likely that the tumor can evade immune recognition by loss of antigen expression. However, there are limitations of fusion proteins as target antigens. The fusion sites may give rise only to peptides that bind strongly to a few MHC molecules. Moreover, even if peptides derived from sequences surrounding the fusion site are identified that bind to MHC, it is essential that these peptides are generated by proteosomal cleavage, bind to the MHC molecules in the ER, and be displayed at the surface of leukemic cells for T-cell recognition.

Studies of the bcr/abl fusion site are the most advanced and have provided provocative data. CD4+ T cells specific for bcr/abl fusion peptides presented by a variety of class II MHC alleles including DR4, DRB1*0901, and DRB5*0101 have been described (Yasukawa et al., 2001, Blood 98:1498-1505; ten Bosch et al., 1999, Blood 94:1038-1045). Peptides spanning the bcr/abl fusion junction have been identified that bind to the HLA-A3, -A11, and -B8 class I molecules (Bocchia et al., 1996, Blood 87:3587-3592). These bcr/abl peptides have been used in vitro to elicit reactive T cells that recognize peptide-pulsed target cells. What has been less clear is whether CML cells actually present bcr/abl peptides at the cell surface. This issue has now been partially addressed by the following. Peptide mixtures eluted from HLA-A3 molecules at the surface of primary CML cells were analyzed by mass spectrometry, and a peptide derived from the bcr/abl junction was identified, providing direct evidence that leukemic cells can process and present bcr/abl derived peptides to CD8+ T cells (Clark et al., 2001, Blood 98:2887-2893). These data provide a rationale for attempting to establish bcr/abl reactive T-cell responses in vivo in CML patients either by vaccination or by adoptive cell therapy (Pinilla-Ibarz et al., 2000, Blood 95:1781-1787).

A second category of proteins considered to be potential targets for immunotherapy are nonmutated proteins that are overexpressed or preferentially expressed in leukemic cells compared with normal cells. The rationale for investigating such proteins as targets for leukemia-specific T-cell therapy comes largely from studies of solid tumors. In melanoma, normal proteins including tyrosinase, gp100, gp75, and MART1, which are involved in melanocyte differentiation, and cancer-testes antigens including the MAGE proteins, which have limited expression in normal tissues, have been identified as targets for tumor-specific T cells. Similarly, in leukemic cells, normal leukaemia associated proteins that are not mutated have been shown to contain epitopes recognized by CD8+ T cells. A few examples of such proteins in leukaemia include proteinase-3, WT-1, hdm2, and human telomerase reverse transcriptase (hTERT). In most cases, these proteins have also been suggested to contribute to the malignant phenotype.

WT-1 is a zinc finger transcription factor that was initially thought to be a tumor suppressor based on studies in Wilms' tumor. However, subsequent studies showed that WT-1 was overexpressed in many malignancies, and it has been implicated in maintaining the malignant phenotype. WT-1 is expressed in normal cells in the kidney, testes, ovary, uterus, and lung, and it is expressed at low levels in normal CD34+ hematopoietic cells (Gaiger et al., 2000, Blood 96:1480-1489). High levels of expression of WT-1 are observed in AML, ALL, and CML, and it has been used as a molecular marker to detect relapse of leukemia. Recent studies have suggested WT-1 may be a suitable target for cellular immunotherapy of leukemia. The sequence of WT-1 was scanned for peptides that bind to class I molecules and peptides that bind to HLA-A2 and -A24 were identified (Oka et al., 2000, Immunogenetics 51:99-107; Ohminami et al., 2000, Blood 95:286-293). These peptides have been used to elicit T cells reactive with WT-1 in vitro. vWT-1 specific T cells have antileukemic activity in vitro and eliminate leukemic progenitors in immunodeficient mice engrafted with human leukaemia (Gao et al., 2000, Blood 95:2198-2203).

Telomerase is a ribonucleoprotein enzyme that is required to maintain telomere length and plays a role in cellular replicative life-span. Human telomerase reverse transcriptase (hTERT) is one component of the complex and is highly expressed in most tumor cells including leukemia. Peptides in hTERT that bind to HLA-A2 and -A24 were used to pulse antigen-presenting cells and isolate T cell lines and clones that recognize tumor cells expressing high levels of endogenous hTERT (Vonderheide et al., 1999, Immunity 10:673-679; Arai et al., 2001, Blood 97:2903-2907). Preliminary studies suggest that hTERT-specific CTLs do not recognize normal hematopoietic cells in vitro, although the more rigorous evaluation of effects on engraftment in NOD/SCID mice have been published (Vonderheide et al., 1999, Immunity 10:673-679).

The human homologue of the mdm-2 oncoprotein, is another self-protein in the category of leukemia-associated proteins that are involved in malignant transformation. Mdm-2 is overexpressed in a variety of malignancies and inactivates the p53 tumor suppressor protein. In contrast to proteinase-3,WT-1, and hTERT, the use of peptides derived from mdm-2 and predicted to bind to class I has not been successful in eliciting mdm-2 reactive T cells, suggesting that tolerance to this normal protein is more complete (Stanislawski et al., 2001, Nat. Immunol. 2:962-970). However, high-avidity T cells specific for mdm-2 can be elicited by immunizing HLA-A2 transgenic mice with mdm-2 peptides or by stimulating T cells from HLA-A2 donors with HLA-A2+ cells pulsed with mdm-2 peptide. The Tcell-receptor α and β genes were cloned from such high-avidity T cells and introduced into normal T cells from HLA-A2+ donors to engineer T cells that are reactive with mdm-2+ tumor cells for potential use in adoptive immunotherapy (Stanislawski et al., 2001, Nat. Immunol. 2:962-970).

An alternative or potentially complementary approach to the adoptive transfer of effector T cells that react with leukemia-associated antigens is to elicit responses in vivo by vaccination. While this approach may be easier to apply more broadly, it has limitations including the potential for toxicity if self-proteins are targeted and the difficulty inducing sufficiently strong T-cell responses to eliminate an established tumor burden. Adoptive transfer studies should assist in defining antigens that can be targeted safely, and the investigation of novel vaccine delivery methods may identify strategies to induce sufficiently potent responses to be therapeutically effective.

Immunotherapy of human cancer has shown limited success to date. This may be due to tumor escape from immune recognition by downregulation of target antigen or antigen-processing machinery (Hui et al., 1984, Nature 311:750-752; Kaklamanis et al., 1992, Int. J. Cancer 51:379-385; Restifo et al., 1993, J. Exp. Med. 177:365-272; Maeurer et al., 1996, J. Clin. Invest. 98:1633-1641), by down-modulation of recognition and stimulation molecules (Matulonis et al., 1995, Blood 85:2507-2515; Munro, 1994, Blood 83:793-798), or because of the production of inhibitory cytokines (Richter et al., 1993, Cancer Res. 53:4134-4137). Antigen-specific T cell tolerance through self-tolerance pathways has also been demonstrated, mostly in animal models and rarely in humans (Bogen et al., 1996, Eur. J. Immunol. 26:2671-2679; Speiser et al., 1997, J. Exp. Med. 186:645-653; Staveley-O'Carroll et al., 1998, Proc. Natl. Acad. Sci. U.S.A. 95:1178-1183; Lee et al., 1999, Nat. Med. 5:677-685). The ability to study tolerance mechanisms in humans has been limited by the small number of well-defined tumor antigens and by the difficulty of detecting tumor antigen-specific T cell responses. Recently, however, an increasing number of human tumor-associated antigens have been identified (Pardoll, 2002, Nat. Rev. Immunol. 2:227-238), and the development of peptide/MHC tetramers has enabled closer study of the immune responses against those antigens (Altman et al., 1996, Science 274:94-96).

PR1 is a nine-amino acid self-peptide derived from proteinase 3 that binds HLA-A2 as a leukemia-associated cytotoxic T lymphocyte (CTL) antigen (Molldrem et al., 1996, Blood 88:2450-2457). PR1-specific CTLs from healthy donors and from patients with chronic myelogenous leukemia (CML) selectively kill CML cells and acute myelogenous leukemia (AML) cells and inhibit the growth of CML progenitors proportional to proteinase 3 overexpression in the target cells (Molldrem et al., 1996, Blood 88:2450-2457; Molldrem et al., 1997, Blood 90:2529-2534; Scheibenbogen et al., 2002, Blood 100:2132-2137). PR1/HLA-A2 tetramers have been used to identify an expanded population of PR1-specific CTLs in CML patients, and their presence correlates with a cytogenetic response to IFN treatment (Molldrem et al., 1999, Cancer Res. 59:2675-2681). PR1-specific CTLs (PR1-CTLs) are also present in the peripheral blood of AML patients during chemotherapy-induced remission, and have a memory phenotype (Scheibenbogen et al., 2002, Blood 100:2132-2137). By purifying the PR1-CTLs, it has been showed that these T cells could specifically kill leukemia cells, but not healthy bone marrow cells (Molldrem et al., 2000, Nat. Med. 6;1018-1023; Molldrem et al., 1999, Cancer Res. 59:2675-2681).

Soluble peptide/MHC tetramers can also be used to distinguish CTLs with high and low T cell receptor (TCR) affinity based on fluorescence intensity, providing a method for rapidly identifying these unique CTL populations (Savage et al., 1999, Immunity 10:485-492; Yee et al., 1999, J. Immunol. 162:2227-2234). CTLs with relative high- or low-affinity TCR can be elicited in vitro by coculturing the lymphocytes with low or high concentrations of target antigen, respectively (Alexander-Miller et al., 1998, J. Exp. Med. 188:1391-1399; Alexander-Miller et al., 1996, Proc. Natl. Acad. Sci. U.S.A. 93:4102-4107; Zeh et al., 1999, J. Immunol. 162:989-994). The effector function of the resulting CTLs has been shown to correlate with TCR affinity (Zeh et al., 1999, J. Immunol. 162:989-994). High-affinity CTLs with specificity for gag, an HIV antigen, were induced to undergo apoptosis when stimulated with high-dose peptide antigen in vitro (Alexander-Miller et al., 1996, Proc. Natl. Acad Sci U.S.A. 93:4102-4107), suggesting that a high viral load might lead to clonal deletion of high-affinity HIV-specific CTLs over time. Similarly, CD4⁺ TCR affinity has been shown to be inversely correlated to antigen dose (Rees et al., 1999, Proc. Natl. Acad. Sci. U.S.A. 96:9781-9786), and in a murine model of CD4⁺ T cell autoreactivity to myelin basic protein, prevention of autoimmunity was observed after loss of high-affinity T cells and outgrowth of low-affinity T cells during exposure to high doses of antigen (Anderton et al., 2001, J. Exp. Med. 193:1-11). These studies suggest there is a peripheral control mechanism preventing the expansion of high-affinity autoreactive T cells that is similar to the differential avidity model for central tolerance (Grossman et al., 1992, Proc. Natl. Acad. Sci. U.S.A. 89:10365-10369; Alam et al., 1996, Nature 381:616-620). It is not known whether similar peripheral tolerance mechanisms apply to self-antigens that are also tumor antigens in humans.

Studies of cellular therapy and vaccination for immunotherapy of human leukemia are just beginning. It is anticipated that these efforts will provide insights that will improve the prospects for immunotherapy as a useful therapeutic adjunct to current treatments. Technical and scientific obstacles need to be addressed, but the understanding of the immunologic mechanisms that may be induced to contribute to tumor eradication are now rapidly evolving. The present invention satisfies this need for improving anti-cancer immunotherapy.

### BRIEF SUMMARY OF THE INVENTION

The invention includes a composition comprising an isolated nucleic acid encoding an epitope of an antigen, wherein the antigen is selected from the group consisting of proteinase 3, PRAME, HOX-A9, Meis1, WT1, survivin, telomerase, MAGE 3, and NY-ESO-1.

The invention also includes a composition comprising an isolated epitope of an antigen, wherein the antigen is selected from the group consisting of proteinase 3, PRAME, HOX-A9, Meis1, WT1, survivin, telomerase, MAGE 3, and NY-ESO-1.

In one embodiment, the epitope of an antigen comprises an amino acid sequence that is at least one amino acid less than the full length amino acid sequence of the antigen.

In another embodiment, the epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

In yet another embodiment, the epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

Also included in the invention is a vector comprising the just-mentioned nucleic acid.

The invention also encompasses a cell comprising the just-mentioned nucleic acid. In another aspect, the cell comprises the just-mentioned amino acid. In a further aspect, the cell comprises an epitope of an antigen, where the antigen is selected from the group consisting of proteinase 3, PRAME, HOX-A9, Meis1, WT1, survivin, telomerase, MAGE 3, and NY-ESO-1. In yet another aspect, the cell is a human cell.

The invention includes an isolated antigen-specific T cell generated according to the method comprising, providing a composition comprising a peptide/MHC tetramer, wherein the peptide/MHC tetramer comprises at least one epitope of an antigen, wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen; contacting a population of immune cells with the composition comprising a peptide/MHC tetramer under conditions suitable for formation of a tetramer-T cell complex; and substantially separating the tetramer-T cell complex from the population of immune cells; thereby isolating the antigen-specific T cell.

The invention also includes an antigen-specific T cell that specifically binds to an epitope of the antigen HOX-A9, wherein the epitope is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

The invention also includes an isolated antigen-specific T cell that specifically binds to an epitope of the antigen Meis1, wherein the epitope is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

In yet another aspect, the isolated antigen-specific T cell is a human cell.

The invention also includes a method of isolating an antigen-specific T cell from a population of immune cells, the method comprising providing a composition comprising a peptide/MHC tetramer, wherein the peptide/MHC tetramer comprises at least one epitope of an antigen, wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen; contacting the population of immune cells with the composition comprising the peptide/MHC tetramer under conditions suitable for formation of a tetramer-T cell complex; and substantially separating the tetramer-T cell complex from said population of immune cells; thereby isolating said antigen-specific T cell.

In one aspect, the peptide/MHC tetramer is a monomer conjugated to a physical support. Preferably, the physical support is selected from the group consisting of a microbead, a magnetic bead, a panning surface, a dense particle for density centrifugation, an adsorption column and an adsorption membrane. More preferably, the physical support is selected from the group consisting of a streptavidin bead and a biotinavidin bead.

In another aspect, the tetramer-T cell complex is substantially separated from a population of immune cells using a method selected from the group consisting of fluorescence activated cell sorting (FACS) and magnetic activated cell sorting (MACS). Preferably, the peptide/MHC tetamer is chemically attached to the surface of the T cell.

In yet another aspect, the population of immune cells are derived from a source selected from the group consisting of a leukapheresis product, peripheral blood, lymph node, tonsil, thymus, tissue biopsy, tumor, spleen, bone marrow, cord blood, CD34+ cells, monocytes and adherent cells.

The invention also includes a method of enriching an antigen-specific T cells from a population of immune cells, the method comprising providing a composition comprising a peptide/MHC tetramer, wherein the peptide/MHC tetramer comprises at least one epitope of an antigen, wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen; contacting the population of immune cells with the composition comprising the peptide/MHC tetramer under conditions suitable for formation of a tetramer-T cell complex; and substantially separating the tetramer-T cell complex from the population of immune cells; thereby enriching for the antigen-specific T cell.

Another aspect of the invention includes a method of stimulating an immune response in a mammal comprising, administering to the mammal a composition comprising an isolated nucleic acid encoding an epitope of an antigen, wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen. Preferably, the mammal is a human.

The invention also includes a method of stimulating an immune response in a mammal comprising, administering to the mammal a composition comprising an isolated epitope of an antigen, wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen. Preferably, the mammal is a human.

An another embodiment, the invention includes a method of stimulating an immune response in a mammal comprising, administering to the mammal a cell comprising an isolated nucleic acid encoding an epitope of an antigen, wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen. Preferably, the mammal is a human. Also preferably, the cell is a human cell.

An yet another embodiment, the invention includes a method of stimulating an immune response in a mammal comprising, administering to the mammal a cell comprising an epitope of an antigen, wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen. Preferably, the mammal is a human. Also preferably, the cell is a human cell.

Also included in the invention is a method for treating cancer in a mammal, the method comprising administering a composition to the mammal, wherein the composition comprises a peptide/MHC tetramer comprising at least one epitope of an antigen, further wherein the antigen is selected from the group consisting of proteinase 3, PRAME, HOX-A9, Meis1, WT1, survivin, telomerase, MAGE 3, and NY-ESO-1, further wherein the epitope comprises an amino acid sequence that is at least one amino acid less than the full length amino acid sequence of the antigen. Preferably, the mammal is a human.

In a further aspect, the cancer is selected from the group consisting of melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, pancreatic cancer, esophageal cancer, brain cancer, lung cancer, ovarian cancer, cervical cancer, multiple myeloma, hepatoma, acute lymphoblastic leukemia (ALL), acute myelogenous leukemia (AML), chronic myelogenous leukemia (CML), and chronic lymphocytic leukemia (CLL).

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic diagram illustrating the basic steps of ‘reverse immunology’ for the determination of immunogenic epitopes. To refine the number of epitopes to be tested, several algorithms predicting peptides that are processed and presented are used. Subsequently, processing and presentation is confirmed experimentally. Only peptides that pass this important test are analyzed further by T-cell repertoire analysis and in vivo models, including HLA-A2 transgenic mice. If all these tests give positive results, the candidate antigen (Ag) can be classified as a tumor Ag to be tested in clinical Phase I trials.

FIG. 2 is a schematic diagram illustrating methods of elucidating the necessary steps to link genomics with cancer immunology for the discovery of novel tumor antigens (Ags). Genes that are overexpressed in cancer cells are identified initially by differential expression analysis, using novel technologies, such as microarray analysis or SAGE. These genes are analyzed for their role in the tumorigenic process. This information is either extracted from literature databases or determined experimentally. Setting priorities is crucial to minimize both time and financial costs of this approach, which could escalate without careful control. Only genes that have been shown to be involved in carcinogenesis or oncogenesis are tested further, using the method of epitope deduction. This includes peptide prediction, binding and presentation,followed by an extensive T-cell repertoire analysis. Abbreviations: CTL, cytotoxic T lymphocyte; ELISPOT, enzyme-linked immunospot; HPLC ESI MS, high-performance liquid chromatography electrospray ionization mass spectrometry; SAGE, serial analysis of Ag expression.

FIGS. 3A and 3B are a series of images depicting patient CD8+ T cell reactivity to various peptide epitopes. The peptide/MHC tetramers used were synthetic, fluorochrome-labeled multimers of MHC molecules bound to a desired peptide antigen that bind in vitro to T cell receptors specific for that peptide-MHC complex. Specific cells were quantified by flow cytometry.

FIG. 4 is a series of images depicting that CD8+ T cells from normal HLA-A2+ donors, stimulated for three rounds in vitro with autologous peptide-loaded antigen presenting cells, demonstrated the induction of CTL specific for Hox-TLD or Mei-AIY (representing 0.4% to 0.7% of CD8+ T cells) which were able to lyse T2 cells loaded with cognate peptide (but not negative control viral peptide). FIG. 4 also illustrates that both Hox-TLD and Mei-AIY specific CTL were able to lyse HoxA9+ and Meis1+leukemia cell lines in an antigen-dependent, MHC-restricted fashion. HoxA9+/Meis1+but HLA-A2-negative leukemia cells were not killed, nor were HLA-A2+ leukemia cells that did not express HoxA9 or Meis1.

FIGS. 5A and 5B are a series of images demonstrating CTL recognition of survivin-expressing tumors through a technique in which human T cells are stimulated in vitro with mRNA electroporated into autologous antigen presenting cells. FIG. 5A illustrates that after two rounds of stimulation, CTL stimulated with full-length survivin mRNA were able to lyse autologous tumor cells expressing survivin in an MHC-restricted fashion. FIG. 5B illustrates that the CTL also mobilized CD107a when incubated with survivin-expressing autologous tumor but not allogeneic survivin-expressing tumor cells mismatched at MHC class I. In HLA-A2+ patients, >80% of CD107a+ CTL in these cultures labeled with the Sur1M2 tetramer whereas <1% of CD107-negative CTL in these cultures were tetramer positive.

FIG. 6 is a table illustrating peptide prediction scores and binding affinity and of HOXa9 and MEIS1 derived peptides to human HLA-A*0201. “BIMAS” and “SYFPEITHI” indicate scores of predicted epitopes in different prediction algorithms. MFI is the Mean Fluorescence Index=Mean Fluorescence T2 (pulsed)−Mean Fluorescence T2 (unpulsed)/Mean Fluorescence T2 (unpulsed). Results are compared to known binding RTPOL peptide numbers. MFI (6h) indicates the Mean Fluorescence Index 6h after peptide withdrawal.

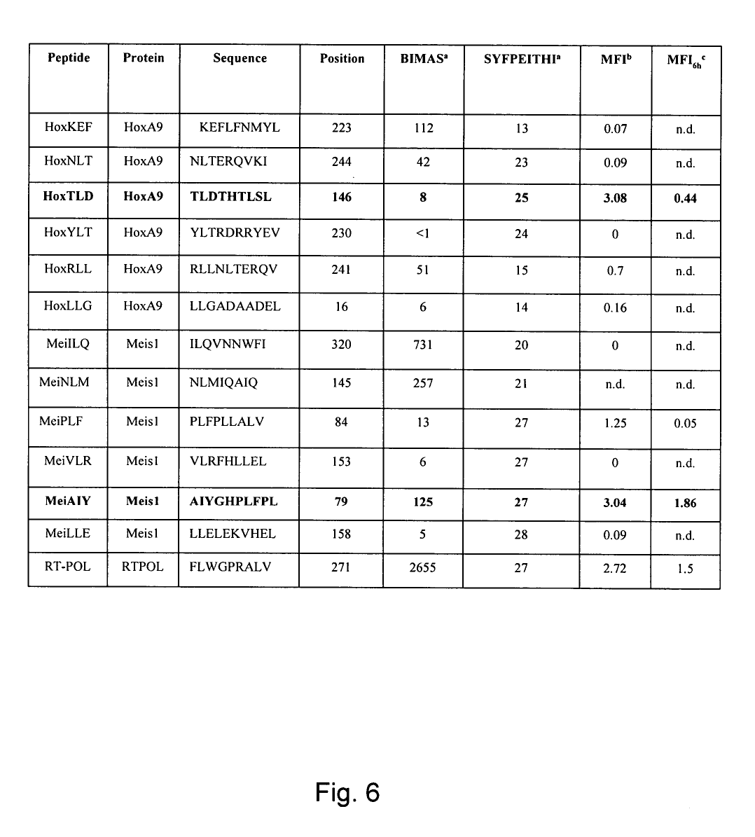

### DETAILED DESCRIPTION

The present invention encompasses compositions and methods for activating, stimulating, isolating and expanding antigen-specific T cells. Preferably, the T cell is a CD8+ T cell. The present invention also includes compositions and methods for the treatment and prevention of cancer, infectious diseases, autoimmune diseases, immune disfunction related to aging, or any other disease state where antigen-specific T cells are desired for treatment.

The invention relates to the observation that myeloid leukemia patients exhibited remission of the disease following receipt of an allogeneic stem cell transplantation. It is believed that the remission involves the induction of a competent anti-leukemia immune response. That is, the invention relates to the identification of an antigen-specific T cell responsible for the remission, as well as to the corresponding antigens and epitopes recognized by the antigen-specific T cell.

In another aspect, the invention relates to the use of a peptide/MHC tetramer (i.e. an HLA-class I tetramer) to identify candidate antigens and epitopes associated with leukemia and other neoplastic or autoimmune diseases.

The invention encompasses compositions and methods useful for treating a patient having a disease, disorder or condition associated with leukemia and other neoplastic or autoimmune diseases. The antigens and epitopes of the invention can be used to develop active vaccines and adoptive immunotherapy. In one embodiment, the peptide/MHC tetramer can be used to activate a T cell. In yet another embodiment, the peptide/MHC tetramer can be used to sort an antigen-specific T cell, where the sorted T cell can be expanded in vitro for use in adoptive immunotherapy. The invention also relates to the use of the novel peptide/MHC tetramer platform technology for diagnostic and prognostic purposes.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

“Allogeneic” refers to a graft derived from a different animal of the same species.

“Alloantigen” is an antigen that differs from an antigen expressed by the recipient.

As used herein, “amino acids” are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988; Houston et al., 1988; Bird et al., 1988).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded soley by a full length nucleotide sequence of a gene. It is readily apparent that the present invention includes, but is not limited to, the use of partial nucelotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“An antigen presenting cell” (APC) is a cell that is capable of activating T cells, and includes, but is not limited to, monocytes/macrophages, B cells and dendritic cells (DCs).

The term “dendritic cell” or “DC” refers to any member of a diverse population of morphologically similar cell types found in lymphoid or non-lymphoid tissues. These cells are characterized by their distinctive morphology, high levels of surface MHC-class II expression. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune precesses.

The term “autoimmune disease” as used herein is defined as a disorder that results from an autoimmune response. An autoimmune disease is the result of an inappropriate and excessive response to a self-antigen. Examples of autoimmune diseases include, but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “DNA” as used herein is defined as deoxyribonucleic acid.

“Donor antigen” refers to an antigen expressed by the donor tissue to be transplanted into the recipient.

“Recipient antigen” referes to a target for the immune response to the donor antigen.

As used herein, an “effector cell” refers to a cell which mediates an immune response against an antigen. An example of an effector cell includes, but is not limited to a T cell and a B cell.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

By the term “effective amount”, as used herein, is meant an amount that when administered to a mammal, causes a detectable level of T cell response compared to the T cell response detected in the absence of the compound. T cell response can be readily assessed by a plethora of art-recognized methods. The skilled artisan would understand that the amount of the compound or composition administered herein varies and can be readily determined based on a number of factors such as the disease or condition being treated, the age and health and physical condition of the mammal being treated, the severity of the disease, the particular compound being administered, and the like.

As used herein, the term “exogenous” refers to any material introduced from or produced outside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide of the present invention can be an epitope.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The “HLA class I (or equivalent molecule)” as used herein is defined as a HLA class I protein or any protein which is equivalent to a human HLA class I molecule from any other animal, particularly a vertebrate and especially a mammal. For example it is well known that in a mouse, the MHC class I proteins are similar in structure to, and fulfill a similar role to, the human HLA class I proteins. Equivalent proteins to human HLA class I molecules can be readily identified in other mammalian species by a person skilled in the art, particularly using molecular biological methods.

The term “helper Tcell” as used herein is defined as an effector Tcell whose primary function is to promote the activation and functions of other B and T lymphocytes and or macrophages. Most helper T cells are CD4 T-cells.

The term “heterologous” as used herein is defined as DNA or RNA sequences or proteins that are derived from the different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

As used herein, “immunogen” refers to a substance that is able to stimulate or induce a humoral antibody and/or cell-mediated immune response in a mammal.

The term “immunoglobulin” or “Ig”, as used herein is defined as a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “major histocompatibility complex”, or “MHC”, as used herein is defined as a specific cluster of genes, many of which encode evolutionarily related cell surface proteins involved in antigen presentation, which are among the most important determinants of histocompatibility. Class I MHC, or MHC-I, function mainly in antigen presentation to CD8 T lymphocytes. Class II MHC, or MHC-II, function mainly in antigen presentation to CD4 T lymphocytes.

As used herein, a “peptide/MHC tetramer” binds to an antigen-specific TCR. The complex comprising a peptide/MHC tetramer and an antigen-specific TCR is refered herein as a “tetramer-T cell complex.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “polypeptide” as used herein is defined as a chain of amino acid residues, usually having a defined sequence. As used herein the term polypeptide is mutually inclusive of the terms “peptide” and “protein”.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

As used herein, “specifically binds” refers to the fact that a first composition binds preferentially with a second composition and does not bind in a significant amount to other compounds present in the sample.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are culture in vitro. In other embodiments, the cells are not cultured in vitro.

As the term is used herein, “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances.

The term “T-cell” as used herein is defined as a thymus-derived cell that participates in a variety of cell-mediated immune reactions.

As used herein, “T cell receptor (TCR)” refers to a surface protein of T cell that allows the T cell to recognize an antigen including an epitope thereof. A TCR functions to recognize an antigenic determinant and to initiate an immune response. A TCR also allows a T cell to recognize an infected cell.

The term “B-cell” as used herein is defined as a cell derived from the bone marrow and/or spleen. B cells can develop into plasma cells which produce antibodies.

As used herein, a “therapeutically effective amount” is the amount of a therapeutic composition sufficient to provide a beneficial effect to a mammal to which the composition is administered.

As used herein, to “treat” means reducing the frequency with which symptoms of a disease (i.e., viral infection, tumor growth and/or metastasis) are experienced by a patient.

The phrase “under transcriptional control” or “operatively linked” as used herein means that the promoter is in the correct location and orientation in relation to a polynucleotide to control the initiation of transcription by RNA polymerase and expression of the polynucleotide.

The term “vaccine” as used herein is defined as a material used to provoke an immune response after administration of the material to a mammal.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “virus” as used herein is defined as a particle consisting of nucleic acid (RNA or DNA) enclosed in a protein coat, with or without an outer lipid envelope, which is capable of replicating within a whole cell.

“Xenogeneic” refers to a graft derived from an animal of a different species.

Description

The invention relates to the identification of candidate antigens and epitopes associated with leukemia and other neoplastic or autoimmune diseases. Using tetramer-guided flow cytometry to evaluate a blood sample from a myeloid leukemia patient, particularly in myeloid leukemia patients who are in remission after receiving an allogeneic bone marrow transplant, it was observed that some patients contained leukemia specific CD8+T cells. These T cells exhibited antigens including, but not limited to proteinase 3, PRAME, HOX-A9, Meis1, WT1, survivin, telomerase, MAGE 3, and others. Based on the present disclosure, T cells specific for these antigens and epitopes therefrom are responsible for inducing and/or maintaining remission in such patients.

The leukemia-associated antigens and epitopes identified herein can be used to develop active vaccines and be applied to adoptive immunotherapy. Further, the present invention includes a tetramer-based platform useful for diagnostic and prognostic purposes.

Identification of a New Class of Tumor Antigens and Epitopes

The present invention relates a novel method for identifying candidate tumor antigens and epitopes therefrom. The method includes the use of available databases based on the human genome project and available bioinformatics tools. Such databases and tools in combination with the disclosure herein provides a method for screening any given protein for immunogenic epitopes.

The method of identifying a tumor antigen and epitopes thereof includes the initial step of identifying a candidate gene corresponding to a tumor antigen and obtaining the predicted candidate peptides thereof using the methods disclosed herein. The predicted peptide is then confirmed experimentally using the methods herein to assess the following characteristics: 1) MHC binding and complex stability of the predicted peptide, 2) MHC presentation of candidate peptides, and 3) T cell repetorie analysis, i.e. characterizing the T cell immune response with respect to the candidate peptide.

Any gene product can be subjected to this analysis without the need to dissect anti-tumor immune responses from cancer patients. Therefore, the present invention provides a method of identifying candidate antigens in diseases in which patient immunoreactivity is weak/absent or when a tumor sample from a cancer patient is not abundantly available.

In any event, the candidate antigen must: (1) include peptide sequences that bind to MHC molecules; (2) be processed by tumor cells such that Ag-derived peptides are available for binding to MHC molecules; (3) be recognized by the T cell repetoire in an MHC-restricted fashion; and (4) permit the expansion of functional T cell precursors that bear peptide-specific T cell receptors.

Identification of and use for such antigens and epitopes is now described in detail herein.

I. Compositions

The present invention encompasses antigens, including derivatives, variant forms or portions thereof, and epitopes thereof. The antigens encompassed by the present invention include, but are not limited to proteinase 3, PRAME, HOX-A9, Meis1, WT1, survivin, telomerase, MAGE 3, NY-ESO-1 and the like.

In another embodiment, the invention encompases novel epitopes (also refered herein as peptides) corresponding to antigens including, but not limited to proteinase 3, PRAME, HOX-A9, Meis1, WT1, survivin, telomerase, MAGE 3, NY-ESO-1 and the like. The epitope of the invention can be in a form of a peptide, preferably, these are the same. As such, a peptide comprising the epitope of the present invention may range in size from about 3-20 amino acids. Preferably, the range is about 4-18 amino acids, more preferably about 5-16 amino acids, and even more most preferably 6-14 amino acids, more preferably about 7-12, and most preferably about 8-10 amino acids.

In yet another aspect of the invention, the epitope comprises an amino acid sequence that is at least one amino acid less than the full length amino acid sequence of the antigen.

The invention relates to the discovery of novel epitopes within an antigen associated with leukemia and other neoplastic or autoimmune diseases. One skilled in the art would recognize that a composition having at least the peptide of the invention is useful for the methods disclosed herein. Therefore, the invention includes a peptide, wherein the peptide does not include the full amino acid sequence of the corresponding antigen in its entirety. For example, the peptide of the invention comprises an amino acid sequence that is at least one amino acid less than the full length amino acid sequence of the antigen.

In a preferred embodiment, an epitope corresponding to the HOX-A9 antigen is selected from the group consisting of HoxKEF, HoxNLT, HoxTLD, HoxYLT, HoxRLL and HoxLLG, which corresponds to the amino acid sequence set forth in SEQ ID NO:1, 2, 3, 4, 5 and 6, respectively. As discussed elsewhere herein, the invention should not be limited to only the sequences set forth in SEQ ID NOs: ID NO:1, 2, 3, 4, 5 and 6. Rather, the invention should encompasss a peptide comprising at least one of SEQ ID NOs: ID NO:1, 2, 3, 4, 5 and 6, wherein the peptide does not include every amino acid corresponding to the HOX-A9 antigen in its entirety; the full-length amino acid sequence of HOX-A9 is set forth in SEQ ID NO: 14, the corresponding DNA sequence is set forth in SEQ ID NO:15. For example, the peptide can comprise any of the SEQ ID NOs: ID NO:1, 2, 3, 4, 5 and 6, wherein the peptide is missing at least one amino acid from the entire amino acid sequence of HOX-A91

In yet another embodiment, an epitope corresponding to the Meis I antigen is selected from the group consisting of MeiILQ, MeiNLM, MeiPLF, MeiVLR, MeiAIY, MeiLLE, which corresponds to the amino acid sequence set forth in SEQ ID NO: 7, 8, 9, 10, 11 and 12, respectively. Again, the invention should not be limited to only the sequences set forth in SEQ ID NOs: ID NO:7, 8, 9, 10, 11 and 12. Rather, the invention should encompasss a peptide comprising at least one of SEQ ID NOs: ID NO:7, 8, 9, 10, 11 and 12, wherein the peptide does not include every amino acid corresponding to the Meis1 antigen in its entirety; the full-length amino acid sequence of Meis1 is set forth in SEQ ID NO:16, the corresponding DNA sequence is set forth in SEQ ID NO:17. For example, the peptide can comprise any of the SEQ ID NOs: ID NO:7, 8, 9, 10, 11 and 12, wherein the peptide is missing at least one amino acid from the entire amino acid sequence of Meis1.

The present invention also provides for analogs of proteins or peptides which comprise an epitope of the present invention. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “derivatives,” and “variants” of the peptides of the invention (or of the DNA encoding the same) which derivatives and variants encompass alteration in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the peptide of the present invention.

The skilled artisan would understand, based upon the disclosure provided herein, that the biological activity of the peptides of the invention encompass, but is not limited to, the ability of a molecule to elicit an immune response. Preferably, the biological activity is the ability for the peptide to induce activation of an immune cell.

Isolated Nucleic Acids

The present invention also includes an isolated nucleic acid encoding an epitope of antigen or derivative/fragment thereof, wherein the epitope comprises the peptides as set forth in SEQ ID NOs:1-12. As discussed elsewhere herein, the peptide of the invention does not include the full amino acid sequence of the corresponding antigen in its entirety. For example, the peptide of the invention comprises an amino acid sequence that is at least one amino acid less than the full length amino acid sequence of the antigen.

In a preferred embodiment, the isolated nucleic acid encodes an epitope corresponding to the HOX-A9 antigen, wherein the epitope is selected from the group consisting of HoxKEF, HoxNLT, HoxTLD, HoxYLT, HoxRLL and HoxLLG, which corresponds to the amino acid sequence set forth in SEQ ID NO:1, 2, 3, 4, 5 and 6, respectively.

In yet another embodiment, the isolated nucleic acid encodes an epitope corresponding to the Meis1 antigen, whrein the epitope is selected from the group consisting of MeiILQ, MeiNLM, MeiPLF, MeiVLR, MeiAIY, MeiLLE, which corresponds to the amino acid sequence set forth in SEQ ID NO: 7, 8, 9, 10, 11 and 12, respectively.

The isolated nucleic acid of the invention should be construed to include an RNA or a DNA sequence encoding an epitope of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The present invention should not be construed as being limited solely to the nucleic and amino acid sequences disclosed herein. Once armed with the present invention, it is readily apparent to one skilled in the art that other nucleic acids encoding the epitopes of the present invention can be obtained using methods known in the art or otherwised described herein (e.g., site-directed mutagenesis, frame shift mutations, and the like).

Further, any other number of procedures may be used for the generation of derivative or variant forms of the antigens of the present invention using recombinant DNA methodology well known in the art such as, for example, that described in Sambrook et al. (2001, Molecular Cloning: A Laboratory Manual, Cold Spring Harbor Laboratory Press, New York) and Ausubel et al. (1997, Current Protocols in Molecular Biology, Green & Wiley, New York).

Procedures for the introduction of amino acid changes in a protein or polypeptide/peptide by altering the DNA sequence encoding the polypeptide/peptide are well known in the art and are also described in Sambrook et al. (2001, supra); Ausubel et al. (1997, supra).

Vectors and Genetically Modified Cells

In other related aspects, the invention includes an isolated nucleic acid encoding an epitope, wherein the epitope is operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

In another aspect, the invention includes a vector comprising an isolated nucleic acid encoding an epitope. Preferably, the epitope is a portion of an antigen selected from the group consisting of proteinase 3, PRAME, HOX-A9, Meis1, WT1, survivin, telomerase, MAGE 3, NY-ESO-1 and the like.

The isolated nucleic acid encoding an epitope of the invention can be cloned into a number of types of vectors. However, the present invention should not be construed to be limited to any particular vector. Instead, the present invention should be construed to encompass a wide plethora of vectors which are readily available and/or well-known in the art. For example, an isolated nucleic acid encoding an epitope of the invention can be cloned into a vector including, but not limited to a plasmid, a phagemid, a phage derivative, an animal viruse, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the epitope, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.”Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the epitope, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means. It is readily understood that the introduction of the expression vector comprising the polynucleotide of the invention yields a silenced cell with respect to a cytokine signaling regulator.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To generate a genetically modified cell, any DNA vector or delivery vehicle can be utilized to transfer the desired polynucleotide to the cell, either in vitro or in vivo. In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

The use of lipid formulations is contemplated for the introduction of the inhibitor of cytokine signaling regulator of the present invention, into host cells (in vitro, ex vivo or in vivo). In a specific embodiment of the invention, the inhibitor may be associated with a lipid. The inhibitor associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid, lipid/tetramer or lipid/expression vector associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes.

Phospholipids may be used for preparing the liposomes according to the present invention and may carry a net positive, negative, or neutral charge. Diacetyl phosphate can be employed to confer a negative charge on the liposomes, and stearylamine can be used to confer a positive charge on the liposomes. The liposomes can be made of one or more phospholipids.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Lipids suitable for use according to the present invention can be obtained from commercial sources. For example, dimyristyl phosphatidylcholine (“DMPC”) can be obtained from Sigma, St. Louis, Mo. Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Phospholipids can form a variety of structures other than liposomes when dispersed in water, depending on the molar ratio of lipid to water. At low ratios the liposome is the preferred structure. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs.

Liposomes interact with cells via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Liposome-mediated oligonucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the lipid may be associated with a hemagglutinating virus (HVJ). This has been shown to facilitate fusion with the cell membrane and promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. In that such expression vectors have been successfully employed in transfer and expression of an oligonucleotide in vitro and in vivo, then they are applicable for the present invention. Where a bacterial promoter is employed in the DNA construct, it also will be desirable to include within the liposome an appropriate bacterial polymerase.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in Drug Carriers in Biology and Medicine, G. Gregoriadis ed. (1979) pp. 287-341, the contents of which are incorporated herein by reference; the method of Deamer and Uster, 1983, the contents of which are incorporated by reference; and the reverse-phase evaporation method as described by Szoka and Papahadjopoulos, 1978. The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

Pulsed Cell

The epitopes of the present invention can be used to activate, otherwise known as pulse or load, an APC. Preferably, the epitope/peptide is used to load an isolated artificial antigen presenting cell (aAPC), termed K562 which is extensively disclosed in US2004/0110290 and US2004/0101519, the contents of which are incorporated by reference as if set forth in its entirety herein.

The pulsed APC can be used to contact a T cell and thereby stimulate the T cell. As such, the invention includes an APC that has been exposed to an epitope/peptide and activated by the epitope/peptide, and as a result capable of stimulating a T cell. An APC may become loaded in vitro, e.g., by culture ex vivo in the presence of the epitope/peptide, or in vivo by exposure to the epitope/peptide.

A skilled artisan would also readily understand that an APC can be “pulsed” in a manner that exposes the APC to a peptide for a time sufficient to promote presentation of that epitope on the surface of the APC. Standard “pulsing” techniques are known in the art (Mehta-Damani et al., 1994; Cohen et al., 1994) and therefore are not dicussed indetail herein.

It is believed that autoimmune diseases result from an immune response being directed against “self-proteins,” otherwise known as autoantigens, i.e., autoantigens that are present or endogenous in an individual. In an autoimmune response, these “self-proteins” are presented to T cells which cause the T cells to become “self-reactive.” According to the method of the invention, APCs are pulsed with a peptide to produce the relevant “self-peptide.” The relevant self-peptide is different for each individual because MHC products are highly polymorphic and each individual MHC molecule might bind different peptide fragments. The “self-peptide” can then be used to design competing peptides or to induce tolerance to the self protein in the individual in need of treatment.

Without wishing to be bound by any particular theory, the peptide in the form of a foreign or autoantigen per se is processed by the APC of the invention in order to retain the immunogenic form of the peptide. The immunogenic form of the peptide implies processing of the antigen through fragmentation to produce a form of the peptide that can be recognized by and stimulate immune cells, for example T cells. The relevant peptide which is produced by the APC may be extracted and purified for use as an immunogenic composition. Peptides processed by the APC may also be used to induce tolerance to the proteins processed by the APC.

The antigen-activated APC, otherwise known as a “pulsed APC” of the invention, is produced by exposure of the APC to a peptide of the invention either in vitro or in vivo. In the case where the APC is pulsed in vitro, the APC is plated on a culture dish and exposed to the peptide in a sufficient amount and for a sufficient period of time to allow the peptide to bind to the APC. The amount and time necessary to achieve binding of the peptide to the APC may be determined by using methods known in the art or otherwise disclosed herein. Other methods known to those of skilled in the art, for example immunoassays or binding assays, may be used to detect the presence of peptide on the APC following exposure to the peptide.

Without wising to be bound by any particular theory, the peptide of the present invention is an antigenic composition, whereby the antigenic composition induces an immune response to the epitope in a cell, tissue or mammal (e.g., a human). As used herein, an “immunological composition” may comprise an epitope (e.g., a peptide or polypeptide), an antigen in the context of an MHC tetramer, a nucleic acid encoding an antigen (e.g., an antigen expression vector), a cell expressing or presenting an antigen or cellular component. In particular embodiments the antigenic composition comprises or encodes all or part of any antigen described herein, or an immunologically functional equivalent thereof. In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

It is understood that an antigenic composition of the present invention may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding a peptide or polypeptide comprising an antigen of the present invention in an in vitro translation system or in a living cell. In addition, an antigenic composition can comprise a cellular component isolated from a biological sample. Preferably the antigenic composition isolated and extensively dialyzed to remove one or more undesired small molecular weight molecules and/or lyophilized for more ready formulation into a desired vehicle. It is further understood that additional amino acids, mutations, chemical modification and such like, if any, that are made will preferably not substantially interfere with the antibody recognition of the epitopic sequence.

A peptide/epitope corresponding to an antigenic determinant of the present invention should generally be at least five or six amino acid residues in length. In addition, the peptides corresponding to the epitopes of the present invention can also range in size from about 3-20 amino acids. Preferably, the range is about 4-18 amino acids, more preferably about 5-16 amino acids, and even more most preferably 6-14 amino acids, more preferably about 7-12, and most preferably about 8-10 amino acids.

In a case where the peptide or polypeptide corresponds to one or more antigenic determinants, the the peptide or polypeptide may contain up to about 10, about 15, about 20, about 25, about 30, about 35, about 40, about 45 or about 50 residues or so. A peptide sequence may be synthesized by methods known to those of ordinary skill in the art, such as, for example, peptide synthesis using automated peptide synthesis machines, such as those available from Applied Biosystems, Inc., Foster City, Calif. (Foster City, Calif.).

Also encompassed in the invention is a composition comprising a peptide or polypeptide corresponding to one or more antigenic determinants in the context of a vaccine. A vaccine of the present invention may vary in its composition. In a non-limiting example, a peptide or polypeptide corresponding to one or more antigenic determinants might also be formulated with an adjuvant. A vaccine of the present invention, and its various components, may be prepared and/or administered by any method disclosed herein or as would be known to one of ordinary skill in the art, in light of the present disclosure.

Longer peptides or polypeptides also may be prepared, e.g., by recombinant means. In certain embodiments, a nucleic acid encoding an antigenic composition and/or a component described herein may be used, for example, to produce an antigenic composition in vitro or in vivo for the various compositions and methods of the present invention. For example, in certain embodiments, a nucleic acid encoding an epitope is comprised in, for example, a vector. The nucleic acid may be expressed to produce a peptide or polypeptide comprising an antigenic sequence. The peptide or polypeptide may be secreted from the cell, or comprised as part of or within the cell.

In a further embodiment of the invention, the APC may be transfected with a vector which allows for the expression of a specific protein by the APC. The protein which is expressed by the APC may then be processed and presented on the cell surface on an MHC receptor. The transfected APC may then be used as an immunogenic composition to produce an immune response to the protein encoded by the vector.

As discussed elsewhere herein, vectors may be prepared to include a specific polynucleotide which encodes and expresses an epitope to which an immunogenic response is desired. Preferably, retroviral vectors are used to infect the cells. More preferably, adenoviral vectors are used to infect the cells.

In another embodiment of this invention, a vector may be targeted to an APC by modifying the viral vector to encode a protein or portions thereof that is recognized by a receptor on the APC, whereby occupation of the APC receptor by the vector will initiate endocytosis of the vector allowing for processing and presentation of the epitope encoded by the nucleic acid of the viral vector. The nucleic acid which is delivered by the virus may be native to the virus which when expressed on the APC encodes viral proteins which are then processed and presented on the MHC receptor of the APC.

As discussed elsewhere herein, various methods can be used for transfecting a polynucleotide into a host cell. The methods include, but are not limited to, calcium phosphate precipitation, lipofection, particle bombardment, microinjection, electroporation, colloidal dispersion systems (i.e. macromolecule complexes, nanocapsules, microspheres, beads, and lipid-based systems including oil-in-water emulsions, micelles, mixed micelles, and liposomes).

In another aspect, a polynucleotide encoding an epitope can be cloned into an expression vector and the vector can be introduced into an APC to otherwise generate an activated APC. Various types of vectors and methods of introducing nucleic acids into a cell are dicussed elsewhere herein. For example, a vector encoding an epitope may be introduced into a host cell by any method in the art. For example, the expression vector can be transferred into a host cell by physical, chemical or biological means. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). It is readily understood that the introduction of the expression vector comprising a polynucleotide encoding an epitope yields a pulsed cell.

In certain embodiments, an immune response may be promoted by transfecting or inoculating a mammal with a nucleic acid encoding an epitope of the invention. One or more cells comprised within a target mammal then expresses the sequences encoded by the nucleic acid after administration of the nucleic acid to the mammal. As such, the present invention includes a vaccine, which may be in the form, for example, of a nucleic acid (e.g., a cDNA or an RNA) encoding all or part of the peptide or polypeptide sequence of an antigenic determinant (i.e. an epitope). Expression in vivo by the nucleic acid may be, for example, by a plasmid type vector, a viral vector, or a viral/plasmid construct vector.

In preferred aspects, the nucleic acid comprises a coding region that encodes all or part of the sequences encoding an antigenic determinant (i.e. an epitope), or an immunologically functional equivalent thereof. Of course, the nucleic acid may comprise and/or encode additional sequences, including but not limited to those comprising one or more immunomodulators or adjuvants.

II. Therapeutic Application

The present invention includes a composition useful for pulsing an APC. The immune response to an antigen presented by an APC maybe measured by monitoring the induction of a cytolytic T-cell response, a helper T-cell response, and/or antibody response to the antigen using methods well known in the art.

The immune response may be an active or a passive immune response. The response may be part of an adoptive immunotherapy approach in which APCs, such as dendritic cells, B cells or moncytes/macrophages, are obtained from a mammal (e.g., a patient), then pulsed with a composition comprising an antigenic composition, and then administering the APC to a mammal in need thereof. The pulsed APC can be contacted with a T cell in vitro to induce T cell activation. Alternatively, the antigenic composition of the present invention can be administered to a mammal to pulse an APC in vivo, whereby the pulsed APC cell can then induce T cell activation in vivo. Therefore, the invention includes a vaccine for ex vivo immunization and/or in vivo therapy in a mammal. Preferably, the mammal is a human.

Ex vivo procedures are well known in the art and are discussed more fully below. Briefly, cells are isolated from a mammal (preferably a human) and activated (i.e., transduced or transfected in vitro) with a vector expressing an epitpe of the present invention or with any other form of the epitope disclosed herein (i.e. chemically synthesized peptide). The pulsed cell can be administered to a mammalian recipient to provide a therapeutic benefit. The mammalian recipient may be a human and the cell so pulsed can be autologous with respect to the recipient. Alternatively, the cells can be allogeneic, syngeneic or xenogeneic with respect to the recipient.

The procedure for ex vivo expansion of hematopoietic stem and progenitor cells described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of DCs comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

A variety of cell selection techniques are known for identifying and separating CD34+ hematopoietic stem or progenitor cells from a population of cells. For example, monoclonal antibodies (or other specific cell binding proteins) can be used to bind to a marker protein or surface antigen protein found on stem or progenitor cells. Several such markers or cell surface antigens for hematopoietic stem cells (i.e., flt-3, CD34, My-10, and Thy-1) are known in the art.

The collected CD34+ cells are cultured with suitable cytokines. CD34+ cells then are allowed to differentiate and commit to cells of the dendritic lineage. These cells are then further purified by flow cytometry or similar means, using markers characteristic of dendritic cells, such as CD1a, HLA DR, CD80 and/or CD86. Following isolation of culturing of DCs, the cells can be modified according to the methods of the present invention. Alternatively, the progenitor cells can be modified prior to being differentiated to DC-like cells.

In any event, an APC can be used to stimulate T cell proliferation in vitro, prior to administering the T cell to a animal, preferably a human. When the T cells expanded using an APC of the invention are administered to an animal, the amount of cells administered can range from about 1 million cells to about 300 billion. Where the APCs themselves are administered, either with or without T cells expanded thereby, they can be administered in an amount ranging from about 100,000 to about one billion cells. The cells may be infused into the animal or may be administered by other parenteral means. The animal is preferably a human patient in need thereof. The precise dosage administered will vary depending upon any number of factors, including but not limited to, the type of animal and type of disease state being treated, the age of the animal and the route of administration.

The APC may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An APC (or cells expanded thereby) may be co-administered to the animal with the various other compounds (cytokines, chemotherapeutic and/or antiviral drugs, among many others). Alternatively, the compound(s) may be administered an hour, a day, a week, a month, or even more, in advance of the APC (or cells expanded thereby), or any permutation thereof. Further, the compound(s) may be administered an hour, a day, a week, or even more, after administration of APC (or cells expanded thereby), or any permutation thereof. The frequency and administration regimen will be readily apparent to the skilled artisan and will depend upon any number of factors such as those already discussed elsewhere herein.

Further, it will be appreciated by one skilled in the art, based upon the disclosure provided herein, that when the APC is to be administered to an animal, the cells maybe treated so that they are in a “state of no growth”; that is, the cells are incapable of dividing when administered to an animal. The cells can be irradiated to render them incapable of growth or division once administered into an animal. Other methods including haptenization (e.g., using dinitrophenyl and other compounds), are known in the art for rendering cells to be administered incapable of growth, and these methods are not discussed further herein.

In addition to using a cell-based vaccine in terms of ex vivo immunization, the present invention also provides compositions and methods for in vivo immunization to elicit an immune response directed against an antigen and/or epitope thereof in a patient.

With respect to in vivo immunization, the antigenic composition is useful for pulsing an APC. Once the APC is activated per se, the antigenic composition can be recognized by a T cell. The recognition of the T cell to the epitope in the context of an APC induces T cell activation. As such, the invention also encompasses the use of pharmaceutical compositions of an appropriate protein or peptide, peptide/MHC tetramer, and/or isolated nucleic acid to practice the methods of the invention.

As used herein, the term “pharmaceutically-acceptable carrier” means a chemical composition with which an appropriate protein or peptide, peptide/MHC tetramer, and/or isolated nucleic acid may be combined and which, following the combination, can be used to administer the protein or peptide, peptide/MHC tetramer, and/or isolated nucleic acid to a mammal.

The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropyl methylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

III. Isolation and Expansion of Antigen-Specific T Cells

The peptide/MHC tetramers of the present invention can be used to take advantage of adoptive immunotherapy around the reinfusion of T cells specific for an antigen/epitope into a patient in need thereof. For example, a peptide/MHC monomer can be conjugated to a physical support (i.e. a streptavidin bead) and therefore provide the opportunity to isolate antigen-specific T cells which when expanded in vitro using conventional methods (i.e. using an APC to active T cells) and those disclosed herein can be used for adoptive immunotherapy. Alternatively, the tetramers of the invention offer the ability to sort antigen-specific T cells using a flow cytometry-based cell sorter. In yet another embodiment of the invention, it is believed that tetramer beads have the potential for directly expanding antigen-specific CD8+ cells in vitro. However, the invention should not be construed to using a peptide/MHC tetramer for only in vitro expansion of T cells, but rather it is envisioned that the peptide/MHC tetramers of the invention can be used to stimulate T cells in vivo.

With respect to using a peptide/MHC tetramer of the invention to sort antigen-specific T cells, this procedure does not require the use of an APC to induce proliferation of the T cell. Rather, a blood sample from a patient can be incubated with the peptide/MHC tetramer to isolate a T cell specific for the peptide/MHC tetramer. The isolated T cell can then be cultured in vitro to generate a desirable number of antigen-specific T cell useful for experimental or therapeutic purposes.

In another aspect of the invention, the peptide/MHC tetramer can be used to isolate an antigen-specific T cell from a T cell population isolated from a blood sample. A T cell population can be obtained using any method known in the art. Preferably, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis or leukapheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Baxter) according to the manufacturer's instructions. After washing, the cells may be resuspended in a variety of biocompatible buffers, such as, for example, Ca⁺²/Mg⁺² free PBS. Alternatively, the undesirable components of the apheresis sample may be removed and the cells are directly resuspended in culture media.

In another embodiment, T cells are isolated from peripheral blood lymphocytes by lysing the red blood cells and by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, CD3+, CD28+ T cells can be positively selected using CD3/CD28 conjugated magnetic beads. In one aspect of the present invention, enrichment of a T cell population by negative selection can be accomplished with a combination of antibodies directed to surface markers unique to the negatively selected cells. A preferred method is cell sorting and/or selection via negative magnetic immunoadherence.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXAMPLES

The invention is now described with reference to the following Examples. These Examples are provided for the purpose of illustration only, and the invention is not limited to these Examples, but rather encompasses all variations which are evident as a result of the teachings provided herein.

The following experiments were conducted to assess for the presence of anti-leukemia specific CD8+ cells and to evaluate whether the cells are functional. The presence of leukemia specific CD8+ cells was demonstrated using a panel of tetramers with specificity for leukemia antigens. Further the present disclosure compares the spectrum of leukemia specific CD8+ T cells between patients pre- and post-allogeneic stem-cell transplantation.

The results herein also address the functional capacity of leukemia specific CD8+ T cells identified by evaluating cytotoxic potential and the ability to secrete cytokines in an antigen-specific manner. Further, the present disclosure demonstrates an approach for expanding leukemia specific CD8+ T cells in vitro for the use in adoptive immunotherapy.

The methods used in the following Examples are as follows:

Preparation of PBMC from Whole Blood

PBMCs are isolated from whole blood from a donor using methods known in the art. Briefly, whole blood is collected from a donor and can be stored at room temperature (RT) overnight if necessary. Alternativley, the whole blood sample can be stored overnight at 4° C. and then warm to RT when ready to use.

The whole blood sample can be spun at 1200 rpm×10 min at RT. Following centrifugation, the top plasma layer is collected and set aside in a labeled tube for use at a later time, leaving about 1 cm of plasma in each tube. The volume of plasma collected is recorded. The blood sample is then reconstituted with RPMI 1640 and pooled together into 50 mL conical tubes and the blood sample is diluted 1:1 with RPMI 1640. 10 mL Ficoll (lymphocyte separation medium) is then pipetted to each 50 mL tube, where 40 mL of blood is overlayed to each tube being careful not to disrupt the Ficoll surface. This mixture is spun for 20 minutes at 2200 rpm. Following centrifugation, 25 mL of the upper plasma layer is removed without disturbing the cell layer. Using a 5 mL pipet, everything from the cell layer is carefully collected, avoiding the RBC pellet at the bottom. The mixture is encubated with RPMI 1640 following a washing stem where the mixture is spun at 1700 rpm×8 minutes at RT. Following centrifugation, the supernatant is gently removed and and washed twice at 1400 rpm×8 minutes in 15 mL conical tubes. The pellet is then resupsend in RPMI 1640 media (with 3% hAB serum, HEPES, L-glutamine, gentamicin) and counted on Coulter. The resulting pellet are PBMCs.

Cryopreservation of PBMC

Following the isolation of PBMCs, the cells can be cryopreserved for use at a later time. Briefly, a Nalgene freezing unit (filled with 2-propanol or 95% ethanol to line indicated) or any freezing container can be used. Freezing Media containing 20% DMSO, 40% hAB Serum and 40% X-VIVO can be used.

The cells are resuspened in in X-VIVO media at 20×10⁶/mL. An equal volume of 2× Freezing Media is added to achieve 10×10⁶/mL. One mL volume of this mixture can be aliquoted chilled cryovials. The cryovials in a chilled nalgene freezing unit are quickly placeed −80° C. The cells are stored −80° C. for one to three days and then transfer to liquid nitrogen for storage.

Ex vivo Tetramer Assay

Cells are washed in T cell media (500 mL RPMI-1640, 50 mL huSerum, 5 mL L-glutamine, 10 mL HEPES, 830 ul of gentamicin). Cells to be tested are resuspend in Staining buffer (filter sterile 1% huSerum in 1×PBS with EDTA [1 mL huSerum+100 mL PBS+200 ul of 0.5M EDTA pH 8.0]) at about 10×10⁶/mL and 100 ul of the mixture is transfered to each well of a 96 well round bottom plate. 0.5 ul of tetramer-PE or tetramer-APC is added to the appropriate wells. The wells are mixed and incubated at 37° C. in a CO₂ incubator for about 10-15 minutes. 150 ul of additional staining buffer is added to each well and the mixture is spun for about three minutes at 1400 rpm. The supernatant is removed using a pipette attached to vacuum apparatus.

A cocktail containing CD8iotest-FITC (1 ul), CD4-PercP (2 ul) and CD14-PercP (2ul) in 100 ul of staining buffer is used for the assay. Table 1 demonstrates how control wells are prepared. The wells are mixed and incubated at 4° C. for 15-20 minutes. Additional 150 ul of the staining buffer is added to each well and spun three times at 1400 rpm. The supernatant is removed using a pipette attached to a vacuum apparatus. The wells are resuspend 100 ul of staining buffer and transferd to FACS tubes containing 200 ul of 2% paraformaldehyde. The samples are analyized using FACS. Samples may be left at 4° C. until analysis.

TABLE 1

| Sample | FITC | PE | PercP | APC | Data | |
|---|---|---|---|---|---|---|
| # | (FL1) | (FL2) | (FL3) | (FL4) | # | Events |
| 1 | IgG | IgG | IgG | IgG | | |
| 2 | CD3 | | | | | |
| 3 | | CD3 | | | | |
| 4 | | | CD3 | | | |
| 5 | | | | CD3 | | |
| 6 | CD8iotest | Tetramer | CD4 + | Tetramer | | |
| | | | CD14 | | | |

Dual CD107a/IFN Assay

T cells are thawed and washed three times in T cell media and then rested at 37° C. in T cell media or IMDM 10% huSerum for one hour. The cells are wased and resuspend at 15.0×10⁶/mL in IMDM 10% huSerum. If cells are from a culture, the cells are wased and place on ice for about 2-3 hours. Following washing of the cells, they are resuspended in IMDM 10% huSerum.

In the first part of the assay, T cells are added into wells of 96 well round bottom plate as desired. 0.5 ul of tetramers is added to the appropriate wells. The plates are incubated at 37° C. for 15 minutes. The cells are then pelleted and resuspend in 100 ul IMDM 10% huSerum.

In the second part of the assay, 20 ul of anti-CD107 is added to each well as appropriate or control IgG (1 ul) is added as a negative control for stimulation. 50 ul of 2×10⁶/mL irradiated T2 cells (10,000 rads) pulsed overnight with peptides is added as described elsewhere herein. T2 cells are prepared by mixing 1 ug/mL peptide+2.5 ug/mL beta2M with 2×10⁶ T2 in 1 mL/well of 24 well plate. T2 cells are incubated with the peptide overnight and and irradiated with 10,000 rads prior to use in the assay.

Anti-CD3/anti-CD28 mixture is prepared by using 6 ul of anti-CD3 of 1 ug/ul to 144 ul media and 1 ul of anti-CD28 of 1 ug/ul to 99 ul media. The cells are incubated with the mixture for 60 minutes at 37° C. in an incubator.

Golgistop solution is prepared by adding 3.2 ul of stock solution to 440 ul media (enough for 22 samples). 20 ul of golgistop is added to each well and the cells were incubated for about 16-20 hours at 37° C. in an incubator. The cells are pelleted and resuspend in 100 ul 1% huS in 1×PBS. The cells can be stained antibodies accordingly at RT for 15 minutes.

Part 3 of the assay includes pelleting the cells following staining the cells with appropriate antibodies. 100 ul per well of BD Cytofix/Cytoperm solution is added to the cells. The cells are incubated at 4° C. for 20 minutes.

1×BD Penn/Wash solution is prepared by diluting the stock solution in distilled HOH at a ration of 1:10. Cells are washed twice in 1×BD Penn/Wash solution (250 ul/well).

50 ul of a 1:100 working solution of IFN-gamma APC in 1×BD Perm/Wash solution is added to the appropriate wells. The cells are incubated at 4° C. for 30 minutes. The cells are washed once in 1×BD Penn/Wash solution (250 ul/well). The cells are then resuspend in Staining Buffer (100ul) and then transfer to FACS tubes and 200 ul of 2% paraformaldehyde is added to the mixture.

Part four of the assay includes the actual analysis of the samples. A brief synopsis of the Dual CD107a/IFN assy is as follows: 1) prepare CD8 cells; 2) stain CD8 cells with tetramers for 15 minutes at 37° C.; 3) wash 1×; 4) add stimulation media; 5) incubate ×1 hour at 37° C.; 6) add golgistop; 7) incubate 16-20 hours at 37° C.; 8) pellet cells; 9) surface stain with CD8 at 4° C. for 15 minutes; 10) wash cells; 11) fix/permeabilize; 12) wash cells; 13) stain for IFN-gamma (1:100) in 50 ul×20-30 minutes at 4° C.; 14) wash cells; and 15) collect cells for analysis.

Proliferation Assay: Evaluate Proliferative Competency of Antigen-Specific CD8+ Cells

This assay is performed to assess the ability of antigen-specific CD8 cells to expand in vitro and uses both an allogeneic and antigen-specific response. This assay can be easily adapted to remove the allogeneic response.

Briefly, 10×10⁶/mL PBMCs are prepared and irradiated at 3300 rads. For irradiated PBMC, use A2+ allogeneic PBMC. 500 ul of PBMC and 500 ul of irradiated PBMC are added to each well. Beta2M (2.5 ul of 1 ug/ul to 100 ul media) is added to wells (100 ul of a 2.5 ug/mL final concentration). The peptide is prepared to an amount of 1 ul (of 1 ug/ul to 100 ul media); 100 ul is added to the cells (1 ug/mL final concentration). The cells are incubate at 37° C. On day one of the asssy, 100 U/mL IL-2 is added to the wells. On day 5, 100 U/mL IL-2 is added to the wells. On day 8, the cells are analyze for antigen-specific CD8+ cells using tetramers.

Flow-Based CTL Assay

The following assay is adapted from Fischer et al. (2002, J. Immunol. Methods 259:159-169). Briefly, T2 cells are labeled as follows. T2 cells are plated at 1×10⁶/mL in serum-free RPMI-1640×1 mL in 24 well plate. 2.5 ug/mL B2M is added to the cells. 1 ug/mL peptide (Tax or Flu) is added to the mixture. The cells are incubate overnight at 37° C. in a CO₂ incubator. The cells are then washed once in RPMI-1640 (serum free). The supernatant is removed; leaving about 25 ul supernatant on the pellet. The remaining cells in suspension are agitated to mix with the supernatant. 1 mL of diluent C is added to resuspend the cells.

Immediately prior to staining, 4×10)(−6)M PKH26 dye is prepared at RT (1:250 dilution of stock; 4 ul of 996 ul diluent C). 1 mL of PKH26 dye is immediately added to 1 mL of cells. The sample is immediatedly mixed by gentle pipetting. The sample is incubate at RT for about 2-5 minutes, with periodical swirling of the tube to insure mixing. The staining reaction is stopped by adding 2 mL of serum in a compatible protein solution. This solution is incubated for 1 minute to stop the staining reaction. The sample is then diluted with 4 mL of complete media (T cell media). The cells are washed two to three times with T cell media.

To assay for the T2 cells, targets at 2e5/mL in IMDM 10% huSerum is prepared. Effectors are prepared at 2×10⁶/mL and 2×10⁵/mL in IMDM 10% huSerum. Using a 96 well round bottom plate, 50 ul of targets are added to each well. The wells are prepare in triplicate as follows (Table 3):

TABLE 3

| Effectors | Targets | Ratio |
|---|---|---|
| 100 ul of 3 × 10⁶/mL | 50 ul of 2 × 10⁵/mL | 30:1 |
| (3 × 10⁵ cells) | (1 × 10⁴ cells) | |
| 33 ul of 3 × 10⁶/mL | 50 ul of 2 × 10⁵/mL | 10:1 |
| (1.0 × 10⁵ cells) | (1 × 10⁴ cells) | |
| 17 ul of 3 × 10⁶/mL | 50 ul of 2 × 10⁵/mL | 5:1 |
| (5 × 10⁴ cells) | (1 × 10⁴ cells) | |
| 33 ul of 3 × 10⁵/mL | 50 ul of 2 × 10⁵/mL | 1:1 |
| (1.0 × 10⁴ cells) | (1 × 10⁴ cells) | |
| 0 | 50 ul of 2 × 10⁵/mL | 0:1 |
| | (1 × 10⁴ cells) | |

The cells are incubated at 37° C. in a CO₂ incubator for 2-3 hours.

The cells are harvested by spinning the cells at 1400 rpm for 3 minutes. The supernatant is removed and the cells are wash twice in 1×PBS (cold). The cells are stained with Annexin V and PI using methods known in the art.

Polyclonal Stimulation of Human CTL with K32-4-1BBL

Cells are maintained in AIM-V 3% huS containing hygromycin B and geneticin (G418). For use in polyclonal stimulation of CD8 cells, K32-4-1BBL cells are irradiated for 10,000 rads. The cells are washed in T cell media (RPMI-1640, 10% huS, HEPES, L-glut, gentamicin). The cells are resuspend at a concentration of 0.5×10⁶ cells/mL. OKT3 (anti-CD3) and anti-CD28 is added to the sample to give a final concentration of 1 ug/mL for each antibody.

K32-4-1BBL cells are incubated with antibodies for 5-10 minutes at RT. K32-4-1BBL cells are added to T cells at ratio of 2:1 T cells:K32. Therefore, in a 24 well plate, about 1×10⁶ T cells+0.5×10⁶ K32-4-1BBL cells are used in a 2 mL sample volume. IL-2 at 20 U/mL and IL-7 at 10 ng/mL is added to the mixture. Addition of IL-2 is every 3-4 days and IL-7 is every 7 days.

T2 Binding Assay

T2 cells are harvested as described elsewhere herin. T2 cells are wash three times in serum free media. T2 cells are prepared to a concentration of 1×10⁶ cells/mL. 1 mL of T2 cells is added to each well of 24 well plate. B2M (1 mg/mL) is added to the mixture to give a final concentration of 2.5 ug/mL. Appropriate control or peptide at 50 ug/mL is added to each wll. The cells are incubated in an incubator at 37° C. For a positive control peptide, RT-POL vs flu or tax can be used.

Next day, cells are harvested and washed three times in serum-free RPMI. The supernatant is removed and the cells are resuspend in 800 ul of serum free IMDM. The cells are plated (400 ul of cells) to one well of a 48 well plate. The cells are incubate for an additional 6 hours in an incubator at 37° C. The remaining cells are saved and place on ice for immediate analysis. The cells are stained for anti-HLA-A2-FITC and for IgG-FITC. The cells are anaylized on a flow cytometer.

The fluorescence index (FI) can be calculated from the mean fluorescence intensity as follows:
FI=(MFI_{peptide}/MFI_{no peptide})−1
The complex's half-life can be calculated using SigmaPlot regression analysis and the following formula:
y=yₒ+a*e^{bx}
t_{1/2}=1/b*ln((y−yₒ)/a) with yₒ=Fiₘₐₓ/2
Conjugation of Monomers to Generate Tetramers

A vial containing 30 ug of monomer is thawed. If necessay, 1×PBS can be added to the monomer to achieve a concentration of 1 mg/mL. Add predetermined amount of streptavidin (APC or PE) to 30 ug of monomer. The sample is placed at room temperature in the dark for 10 minutes. This step is repeated for about three to four times, or as necessary. The sample is diluted to a ration of 1:1 with 1×PBS. 0.5 ul of the conjugated tetramer per 100 ul of cells at 10×10⁶/mL is used for assay.

Preparation of Tetramer Beads

150 ul of dynal streptavidin beads (Dynabeads M-280 streptavidin; prod. No. 112.06) is added to a 10 ug of monomer. The mixture comprising the beads and monomer is incubate for 30 minutes at RT with occasional swirling in an eppendorf. The beads are washed three times with RPMI-1640 without serum by placing the eppendorf on a magnet for 3-5 minutes per wash. The beads are resuspended in 800 ul of RPMI-1640 and store at 4° C. for use at a later time.

Use of Tetramer Beads

Tetramer beads can be used in expanding antigen-specific CD8+ cells. In a 15 mL polypropylene tube, 5 ul of tetramer beads is added to 100 ul of cells (10×10⁶/mL) at RT for 20 minutes in T cell media (RPMI-1640, 10% huS, L-glut, HEPES, gentarnicin). Occasionally, the tube to swirled to insure suspension of the beads. Post incubation, the cells are diluted to a final concentration of 1×10⁶/mL and plated in a 24 or 48 well plate. The cells are incubated with 1 ug/mL anti-CD28 on day 0 and 10 ng/mL IL-15 is added on day 1 and 5.

Tetramer beads can be also used for sorting for antigen-specific CD8+ cells. In a 15 mL polypropylene tube, 3 ul of tetramer beads is added per 1×10⁶ cells at 10×10⁶/mL in FACS staining buffer (1% huSerum 1×PBS with 1:5000 0.5M EDTA pH8.0). The mixture is incubate at RT for 15 minutes. The cells are seperated on a magnet and washed for an additional time using FACS staining buffer. The cells are resuspend in either 100 ul or 500 ul of media and plated into 96 round bottom or 48 well plate.

Production of Monomers for Generation of Tetramers

Large production of a desired monomer can be generated using a bacterial inclusion body. Briefly, a 100 mL culture of the construct from which to make inclusion bodies is started and incubated overnight with appropriate antibiotics. Such a construct is that A2 construct with 100 ug/mL of ampicillin. The culture is grown up in LB Broth. Following an overnight culturing period, the culture is split and cultured with shaking at 37° C. until the OD at 600 nm is between 0.6-0.8.

IPTG is added to the culture to a final concentration of 0.5 mM. (1000× stock is kept at −20°). The culture is allowed to shake at 37° C. for an additional 4 hours. The bacteria is pelleted in an RC-5 centrifuge in large plastic containers. Spun at 3500 rpm in GSA3 rotor for 15 minutes. The supernatant is removed and the bacteria pellet is resuspened in Lysis Buffer (add 1:100 DTT). Approximately 60 mL of lysis buffer is sufficient for 6L bacteria. The mixture and be frozen at −70° C. overnight or for at least 1.5 months.

Purification of bacterial inclusion bodies can be accomplished as follows. The frozen bacterial pellet/lysate is thawed in room temperature. The mixture is transfered to a polypropylene beaker with a clean stir bar. Stirring is at a medium speed while adding dropwise of the following solution (for a 60 ml solution).
a. 300 ul of 1M MgCl2;
b. 600 ul of Triton X-100; (Kept at RT on chemical shelf)
c. 24 ul of 110 U/ul DNase (=2640 units);
d. 1200 ul of lysozyme; and
e. 60 ul of I M DTT (stock in −20 degree C freezer).
   This mixture is mixed for about 10 minutes and then transfered to appropriate tubes for GS-600 rotor to use in RC-5 centrifuge. The mixture is sonicated and subsequently spun at 12.5 K for 12 minutes. The supernatant is removed and 5 mL of Wash Buffer with Triton (add DTT to 1 M final concentration) is added followed by a sonication step (after sonication, 20 mL of Wash Buffer with Triton is added to the mixture), and the subsequent spun. This step is repeated two times or for a desird amount of time.

The pellet is resuspened in urea solution. Keeping the amount to the minimal in order to get the protein into solution. Transfer mixture to a Beckman ultra centrifuge tube (14×89 mm) and spun in the SW 41Ti rotor at 25K for 25 minutes. The supernatant is harvested and the OD is checked at 280 nm.
(O.D.280×DF)/(Extinction coefficient)=mg/mL, where
DF=dilution factor
Extinction coefficient:
   i. Inclusion bodies=2.1
   ii. Complex=1.8
   iii. B2M=1.55.
      Protocol for 100 mL-Scale Refolding Reaction

100 mL of ice cold refolding buffer (100 mM Tris pH 8.0, 400 mM L-Arginine, 2 mM EDTA and 20% glycerol) is placed into a 200 mL autoclaved beaker with a stir bar. While stirring, PMSF (dissolved in 100% isopropanol at 0.2 M stock) is added to a final concentration of 0.2 M (1:1000 dilution, for 100 mL add 100 ul of stock). Fresh powder (153.5 mg) of GSH; glutathione reduced (final 5mM) is added. Fresh powder (30.65 mg) of GSSH, glutathione oxidized (final 0.5 mM) is added. 3 mg of dissolved peptide (20 mg/mL in HPLC-grade DMSO from Sigma) is then added. 2.4 mg of B2M IC (MHC class I heavy chain-B2 microglobulin inclusion bodies) in 1 mL of injection buffer through a 1 mL syringe with a 27½ gauge needle is added. 3.1 mg of A2 IC (MHC class I heavy chain-A2 inclusion bodies) in 1 mL of injection as above is added. The solution is mixed in a cold room overnight.

The next morning, the injection of 3.1 mg of A2 IC is repeated. The solution is again stired in the cold for a period of time. At the end of the day, the injection of 3.1 mg of A2 IC is repeated and the solution is set for stirring overnight in a cold room.

On day three, the sample is concentrated and desalted. The sample is centrifuge in a 50 mL conical tube with a table top centrifuge at 3600 rpm for 20 minutes. The sample is decanted into a clean 50 mL tube and keep on ice. The sample is concentrated down to 5-8 mL with the vivaflow 50 system (30,000 MWCO) using single module setup.

After the sample is at an appropriate concentration, the sample can be biotinylated. Biotin Ligase from Avidity can be used. 30 ug of biotin ligase is added to the sample and allowed to incubated RT overnight.

On day four, the sample is subjected to HPLC for isolation of the refolded tetramer by Superose 12 Gel Filtration. The biotinylation reaction mix is concentrated down to 1 mL in a centricon filter centrifuge tube (table top centrifuge at 3600 rpm). The mixture is concentrated to 1 mL per tube of 2 tubes. The sample is collected into an eppendorf tube and spun in a table top micocentrifuge for 10 minutes at 13,000 rpm. The supernatant is applied to the HPLC Superose gel filtration column using the tetramer method described elsewhere herein.

### Example 1

### Epitope Deduction

Clinically successful specific cancer immunotherapy depends on the identification of tumor regression antigens. Prior to the present disclosure, tumor antigens have been identified by analyzing either the T cell or antibody responses of cancer patients against autologous cancer cells. The unveiling of the human genome, improved bioinformatics tools, and optimized immunological analytical tools have made it possible to screen any given protein for immunogenic epitopes. The results herein demonstrate that based on these advancements, a new class of tumor antigens can be identified by directly linking cancer genomics to cancer immunology and immunotherapy.

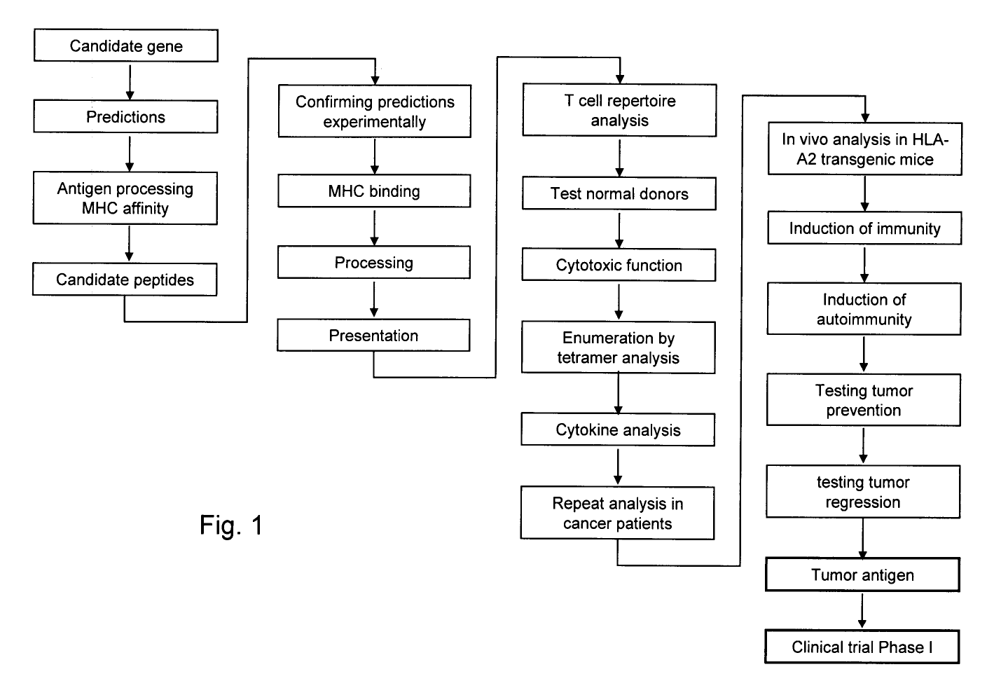

The discovery of these novel tumor antigens are based largely on the method depicted in FIG. 1; namely, the deduction of peptide MHC epitopes from genes having broad overexpression in cancer and known crucial roles in tumor growth and development.

The method of epitope deduction, otherwise known as “reverse immunology,” is based on the fact that candidate T cell peptide epitopes can be identified based on predicted binding affinities of peptide for MHC, and scrutinized for immunogenicity based on the functional capacity of experimentally generated peptide-specific T lymphocytes. Briefly, epitope deduction for the identification of tumor antigens involve the use of several algorithms which are publicly available for predicting peptide affinities to MHC (<<www.bimas.dcrt.nih.gov/molbio/hlabind>>; <www.uni-tuebingen.de/uni/kxi>>; <<www.ludwig.unil.ch/SEREX/mhc_pep.html>>). Peptide prediction for MHC class II epitopes available by way of a software package described in <<www.tepitope.com>>.

Prediction of antigen processing can be accomplished using an algorithm for proteosomal cleavage (PaProC), which is available at <<www.uni-tuebingen.de/uni/kxi>>. No software is yet available that predicts other important steps of Ag processing. It is important to note that these algorithms yeild estimates that need to be validated experimentally.

At this time, there is not a known standard established for the analysis of the necessary steps of the antigen processing machinery. Because there is alternative cleavage between regular proteosomes and the immunoproteosome, it has been suggested that the analysis of proteosomal cleavage further limits the candidates that need to be tested in more laborious T cell screening systems.

Numerous tests have been described to estimate or directly test binding affinity of peptides to MHC molecules. These include cellular assays using transporter associated with Ag processing-deficient T2 hybridoma cells and enzyme linked immunoabsorbent assay-based assays using purified MHC molecules. Because the complex stability between peptide and MHC class I molecule plays an important role for a peptide's immunogenicity, and because complex stability cannot be predicted at this time, it is necessary to determine complex stability experimentally by testing for the MHC binding and complex stability of predicted peptides.

Elution of peptides out of MHC class molecules using technologies such as tandem mass spectrometry (MS/MS) analysis or high-performance liquid chromatography electrospray ionization mass spectrometry (HPLC ESI MS) are direct approaches to identify peptides that are presented by tumor cells. The sensitivity of this methodology can be as high as about 10 fmol of a single low abundance peptide equivalent or <10 copies/cell. Indirectly, the presence of immunogenic epitopes can be assessed using peptide-specific T cell clones and a panel of tumor cell lines transfected either with the right restriction element or the tumor antigen of interest (if this is not endogenously expressed).

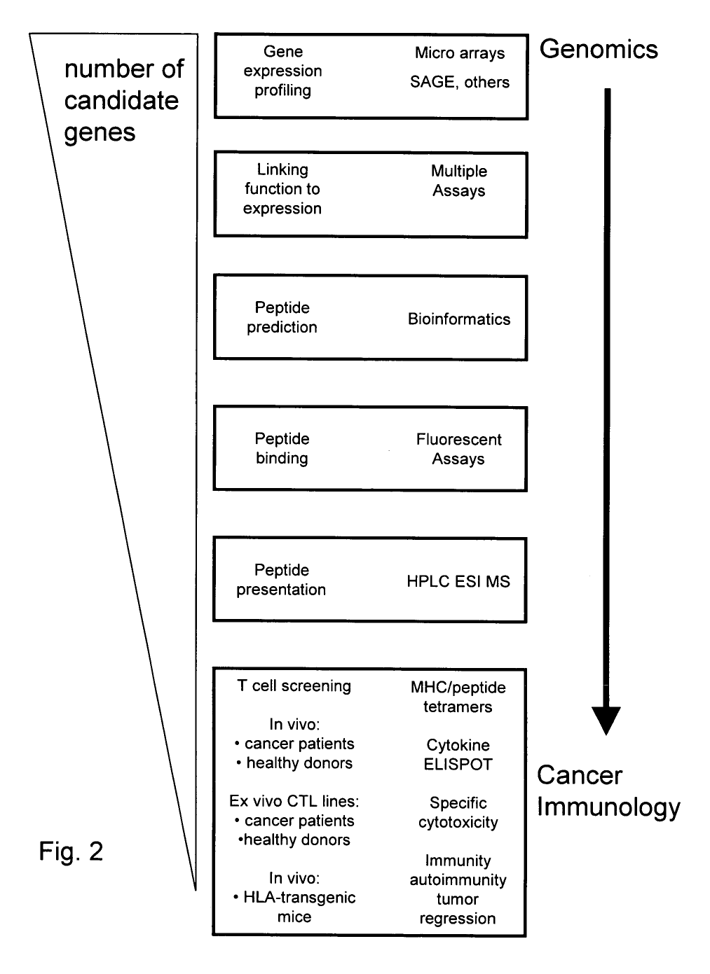

The most important assessment is the quantitative and qualitative analysis of the T cell repertoire specific for any given epitope. FIG. 2 summarizes a potential algorithm for characterizing the T cell immune response.

Candidate peptides are screened systematically against a series of experimental criteria, as shown in FIG. 2. Any gene product can be subjected to this analysis without the need to dissect anti-tumor immune responses from cancer patients. Such dissection is the cornerstone of the classical discovery approach, but has now become a major limitation as strategies in cellular tumor immunology extend beyond melanoma to the majority of common cancers in which patient immunoreactivity is weak or absent.

The candidate antigen has the following characteristics: (1) include peptide sequences that bind to MHC molecules; (2) be processed by tumor cells such that Ag-derived peptides are available for binding to MHC molecules; (3) be recognized by the T cell repertoire in an MHC-restricted fashion; and (4) permit the expansion of functional T cell precursors that bear peptide-specific T cell receptors. Most commonly, MHC class I-restricted candidate epitopes are used to generate specific cytotoxic T lymphocytes that are then evaluated for cytotoxicity of Ag⁺ tumor cells expressing the appropriate MHC allele.

The tumor antigens are chosen based on their role in cancer biology rather than the analysis of the cancer patient's immune responses to these genes. Epitope candidates are then deduced and tested experimentally. This strategy of discovery is suited particularly to determine candidate tumor antigens that are expressed at the earliest steps of tumor formation. Without wishing to be bound by any particular theory, such genes are believed to be ideal targets for treatment strategies in the adjuvant setting or even for preventive immune intervention. The pooling of several such targets, similar to combination chemotherapy or combination antimicrobials, is a goal of antgen-specific immunotherapy.

### Example 2

### Leukemia Specific CD8+ T Cells

It has been observed that patients with AML or CML in remission following alloSCT (allogeneic stem cell transplantation) exhibited significant numbers of peripheral blood cytotoxic T lymphocytes (CTL) that recognize varying combinations of epitopes derived from leukemia-associated antigens. Prior to transplantation or in normal individuals, CD8+ T cells specific for these antigens are rare.

In order to assess the repertoire and function of CD8+ T cell responses after transplantation and determine whether activated donor lymphocyte infusions (aDLI) augments the proliferative and cytoproductive function of these cells in vivo, a method of epitope deduction was used to identify a panel of candidate peptide antigens. The candidate peptide antigens were then screened with patient T cells for reactivity to these antigens using peptide/MHC tetramer technology. Samples were obtained from a total of 12 patients with AML or CML before or after alloSCT. Twenty-one HLA-A2-binding epitopes from 8 candidate antigens were examined. Three HLA-A2-binding epitopes from the viruses HTLV-1, CMV, and influenza were used as negative and positive controls. For the identification of epitopes from candidate tumor-associated antigens, a database analyses was used to (i) select gene products with selective tumor expression and (ii) scan the deduced protein sequence for peptides that match known MHC binding motifs. HLA-A2-binding epitopes were selected because HLA-A2 is the most common HLA class I allele in the tested patients. Although some tumor-associated epitopes used in the assay have been previously described, the results herein discloses some novel antigens and epitopes. For example, novel epitopes were identified in the molecules HoxA9 and Meis1, both of which are linked to leukemogenesis but neither known to express candidate T cell epitopes.

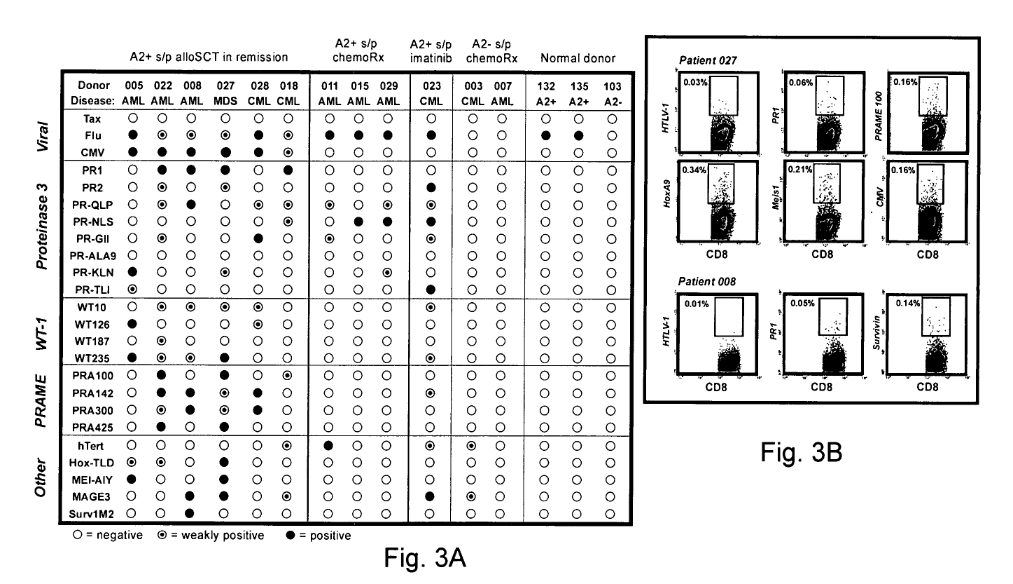

To examine patient T cell reactivity to these peptide epitopes, peptide/MHC tetramers were created for each epitope. Tetramers are synthetic, fluorochrome-labeled multimers of MHC molecules bound to a desired peptide antigen that bind in vitro to T cell receptors specific for that peptide-MHC complex. Specific cells can then be quantified by flow cytometry. Methods for the conjugation of tetramers to various fluorochromes such that the binding of multiple tetramers for the simultaneous examination in a single tube were developed. It was observed that this approach decreased the number of patient cells required for the analysis and increases data output. FIG. 3 summarizes the analysis of patient CD8+ T cell reactivity to the panel of epitopes and demonstrates that T cell reactivity to leukemia-associated antigens in this clinical setting is extensive. Examples of positive hits are shown. Similar results were obtained in one CML patient treated with imatinib and in a second study, lymphoma patients following alloSCT were evaluated. It was observed that CD8+ T cells in these patients responsed to survivin, WT-1 and PRAME antigens.

The next set of experiments employed three tetramer-independent tests to measure in vitro function of these T cells in response to cognate peptide: (i) intracellular IFN-gamma secretion assay, (ii) 7-day proliferation assay, and (iii) CD107a mobilization assay, the latter being a flow-based strategy to detect CD107a on the surface of CTL that have undergone degranulation as part of target killing (Rubio et al., 2003, Nat Med 9:1377-1382). Overall, it was observed that CTL specific for leukemia-associated antigens after alloSCT displayed a decreased to absent functional capacity ex vivo as measured by their ability to secrete IFN-gamma and release of CD107a in an antigen-specific manner. Similarly, poor IFN-gamma and CD107a responses were also observed for T cells specific for viral epitopes. Moreover, CD8+ T cells specific for leukemia-associated or viral antigens were also unable to proliferate to antigen-specific stimuli. This decreased capacity for proliferation was not recovered with the addition of IL-2 or IL-15 and did not correlate with CD28 or CD57 expression.

These data demonstrate that leukemia-specific T cells induced following alloSCT are “seen but not heard”, and the mechanism underlying this silencing may contribute in important ways to the regulation of the graft vs. leukemia effect. Without wishing to be bound by any particular theory, it is believed that T cell function in the immediate months following alloSCT is globally depressed, aggravated in particular by immunosuppressive drugs administered for graft vs. host concerns. However, some of the studies herein were performed on samples not obtained in the first months following alloSCT, including results from one CML patient 5 years after alloSCT whose peripheral T cells functioned poorly in assays herein despite obvious reactivity of these T cells to tetramers. It is also possibile that any potential CTL priming to leukemia antigens that occurs soon after alloSCT occurs in the absence CD4+ T cells, which reconstitute notoriously slowly. Although homeostasis proliferation characteristic of the post-transplant setting may offer advantages for CD8+ T cell priming (Dummer et al., 2002, J Clin Invest 110: 185-192), doing so in the absence of T cell help may lead to the expansion of “helpless” CTL, characterized in some model systems as antigen-specific CTL that can mediate effector functions such as cytotoxicity and cytokine secretion upon restimulation, but do not undergo a second round of clonal expansion (Janssen et al., 2003, Nature 421:852-856).

### Example 3

### HoxA9 and Meis1 as Tumor-Associated Antigens Recognized by CTL

HoxA9 and Meis1 were evaluated as candidate leukemia-associated antigens for two reasons: (i) they are each highly co-expressed in most cases of AML, CML, and MDS (a notable exception is M3 AML), and (ii) each plays a defining and collaborative role in the induction of AML. HoxA9 is considered to be the single most highly correlated gene (out of >6,000) for poor prognosis in human AML and is essential for MLL-dependent leukemogenesis in vivo. HoxA9 cooperatively binds DNA with Meis1 such that only the co-overexpression of both transcription factors results in rapid leukemic transformation of primitive hemopoietic cells. HoxA9 and Meis1 are also normally expressed in bone marrow but otherwise have limited post-natal expression. In 29 primary AML samples tested, it was observed HoxA9 expression in 50% of samples and Meis1 expression in 72% by RT-PCR, although only 2 samples were positive for MLL translocations. Thus, targeting HoxA9 and Meis1 immunologically not only has broad clinical implications but may also provide effective immune targets for which mutation or loss as a means of immune escape is incompatible with sustained tumor growth.

HLA-A2-restricted epitopes derived from HoxA9 and Meis I were predicted using two computational algorithms, and 6 epitopes from each gene product with the highest predicted likelihood of binding to HLA-A2 were evaluated empirically using a flow-based T2 assay, as described (Vonderheide et al., 1999, Immunity 10:673-679). Two peptides (Hox-TLD and Mei-AIY) demonstrated the best binding and MHC complex stability. Using peptide/MHC tetramers for these 2 epitopes, it was found that certain AML and MDS patients had detectable HoxA9- and Meis1-specific CTL in peripheral blood after, but not before, alloSCT (FIG. 3). The same cells in normal HLA-A2+ donors were undetectable.

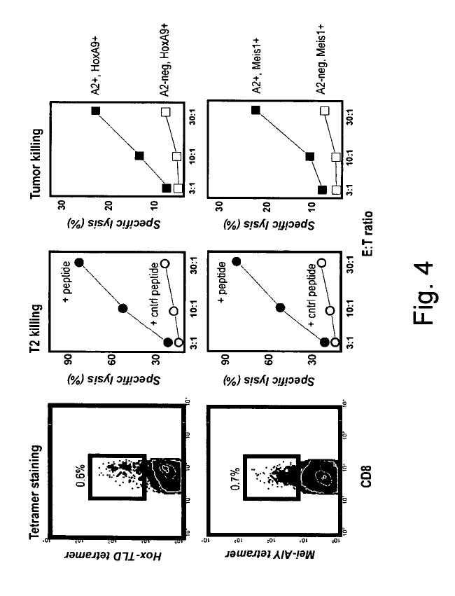

To determine whether these peptides could trigger the expansion of specific CTL, CD8+ T cells from normal HLA-A2+ donors were stimulated in vitro with autologous peptide-loaded antigen presenting cells, using a system previously described (Vonderheide et al., 1999, Immunity 10:673-679). After 3 rounds of stimulation, tetramer analysis demonstrated the induction of CTL specific for Hox-TLD or Mei-AIY (representing 0.4% to 0.7% of CD8+ T cells) which were able to lyse T2 cells loaded with cognate peptide (but not negative control viral peptide) (FIG. 4). Moreover, both Hox-TLD and Mei-AIY specific CTL were able to lyse HoxA9+ and Meis1+leukemia cell lines in an antigen-dependent, MHC-restricted fashion. HoxA9+/Meis1+but HLA-A2-negative leukemia cells were not killed, nor were HLA-A2+ leukemia cells that did not express HoxA9 or Meis1 (FIG. 4). These data suggest that the human T cell repertoire includes specificities for HoxA9 and Meis1 and also imply that Hox-TLD and Mei-AIY are naturally processsed and presented by leukemia cells in the groove of MHC where they can trigger tumor cytoxicity by specific CTL.

### Example 4

### Survivin as a Tumor-Associated Antigen Recognized by CTL

Similarly, the anti-apoptotic protein survivin was evaluated as a candidate leukemia-associated antigen because (i) it is overexpressed in the majority of cases of AML and CML-blast crisis, and (ii) it plays a critical role as a survival factor in cancer cells such that mutation or loss as a means of immune escape may be deleterious to sustained tumor growth. Although survivin was initially appreciated as an efficient inhibitor of apoptosis, it has been shown that survivin also functions to preserve mitotic progression. For many histologies, patients whose tumors express survivin have decreased survival, increased rate of relapse, and increased resistance to therapy. Survivin is strongly expressed during embryogenesis but is absent in terminally differentiated normal tissue. Thymocytes, bone marrow progenitor cells, and basal epithelial cells of the colon are survivin-positive. Several epitopes derived from survivin have been described that bind to MHC class I and can be recognized by CTL that mediate lysis of survivin-positive tumors. Spontaneously occuring CTL specific for survivin have been described in a few patients with CLL or CML (Andersen et al., 2001, Cancer Res. 61:5964-5968). For the HLA-A2-binding epitope Sur1M2, tetramer analysis was used to identify survivin-specific CTL in patients with AML and lymphoma following alloSCT.

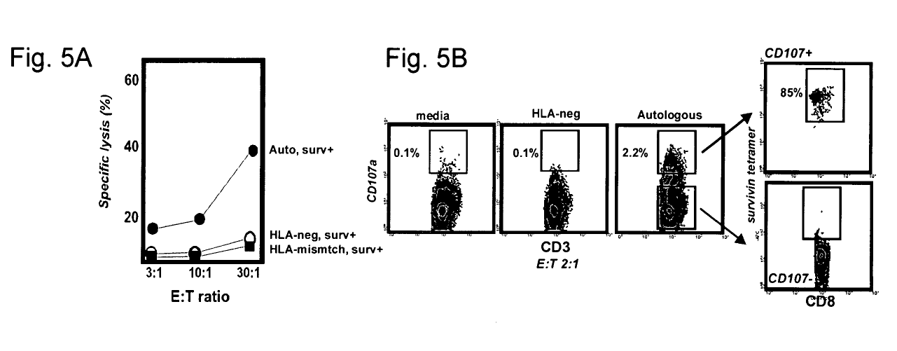

Further experiments were conducted to dissect CTL recognition of survivin-expressing tumors by developing a technology in which human T cells are stimulated in vitro with mRNA electroporated into autologous antigen presenting cells (Coughlin et al., 2004, Blood 103:2046-2054). After two rounds of stimulation, CTL stimulated with full-length survivin mRNA were able to lyse autologous tumor cells expressing survivin in an MHC-restricted fashion (FIG. 5). These CTL also mobilized CD 1 07a when incubated with survivin-expressing autologous tumor but not allogeneic survivin-expressing tumor cells mismatched at MHC class I. In HLA-A2+ patients, >80% of CD107a+ CTL in these cultures labeled with the SurlM2 tetramer whereas <1% of CD107-negative CTL in these cultures were tetramer positive (FIG. 5). These data confirm that MHC class I-restricted CTL can lyse tumor cells in an antigen dependent fasion, and provide evidence for the immunodominance of the Sur1M2 epitope in HLA-A2 patients.

### Example 5

### Clinical and Immunologoical Impact of Peptide Vaccination

A peptide/adjuvant/GM-CSF vaccine formulation can be implemented for peptide vaccination strategies in leukemia. Such a strategy adopts those strategies involving telomerase reverse transcriptase hTERT vaccines. Peptide/MHC tetramer analysis of PBMC demonstrated that 50% of patients responded immunologically to the vaccine, including all those with clinical benefit. Tetramer+ CD8+ cells induced by vaccination lysed telomerase-positive, HLA-A2+ (but not HLA-A2-negative) carcinoma cells. hTERT-specific CD8+ tumor infiltrating lymphocytes were observed by tetramer analysis after, but not before vaccination. These results suggest that peptide vaccination with adjuvant and GM-CSF is highly feasible and can safely induce antigen-specific immune responses that mediate anti-tumor effects in vivo.

### Example 6

### Characterize Anti-Tumor T Cell Immunity

The following experiments were conducted to test the hypothesis that non-polymorphic self-antigens overexpressed by leukemia cells can trigger the proliferation of antigen-specific CD8+ T cells in the post-alloSCT setting. The rationale for this hypothesis derives from the observation that T cell reactivity to candidate leukemia-associated antigens after but not before alloSCT is extensive.

The experiments are perfermed to determine T cell reactivity to leukemia-associated antigens using a panel of candidate peptide epitopes vs. reactivity to total tumor antigen using mRNA from autologous tumor. Two approaches can be used to evaluate anti-leukemia CTL activity in patients with myeloid leukemia. First, CD8+ T cells isolated from blood samples obtained after alloSCT can be analyzed for antigen-specificity using a panel of peptide/MHC tetramers. Initially, a panel of HLA-A2 tetramers encompassing 24 epitopes from 8 candidate leukemia-associated antigens and 3 viral proteins is used. However, based on the present disclosure, the panel of peptide/MHC tetramers can be increased by building tetramers for epitopes from additional antigens (e.g. HoxA7, cytochrome P450 IBI) as well as for epitopes that bind to other HLA alleles (e.g. HLA-A1, -A3, -A24). In any event, samples from patients exhibiting tetramer-reactive CD8 T cells can be studied further for function. To do this, CD8+ T cells from patients can be tested for (i) intracellular IFN-gamma production in response to cognate vs. control peptide, and (ii) in vitro proliferation in response to peptide, using methods discussed herein. CD8+ T cells that proliferate in response to peptide can then be tested for cytotoxicity using either autologous leukemia cells or allogeneic leukemia cell lines matched for the restricting HLA allele. Both chromium release and CD107a mobilization assays can be used. Cytotoxicity can also be tested against a panel of normal cells expressing the candidate antigen, which in many cases can be normal hematopoietic progenitor cells.

In a second assay for CTL activity, RNA transfection technology can be used to analyze antigen-specific CD8 T cell responses without regard to HLA alleles. In this assay, CD40-activated B cells (CD40-B) can be generated as described (Coughlin et al., 2004, Blood 103: 2046-2054) and transfected with autologous leukemia RNA, pooled RNA from multiple allogeneic leukemia samples, or GFP mRNA as a control. Patient T cells stimulated in vitro with tumor RNA-transfected CD40-B cells can then be analyzed for cytoxicity against (i) autologous leukemia cells, and (ii) allogeneic leukemia samples matched for at least one allele (vs. HLA unmatched cells as controls). Among the advantages of using CD40-B cells instead of dendritic cells as APCs in this system is the ability to generate >100 million CD40-B cells from <10 cc of peripheral blood owing to their massive proliferative potential. Tumor RNA-stimulated T cells that exhibit HLA-restricted killing can then be probed with our tetramer panel to dissect potential molecular targets of the CTL, as described (Coughlin et al., 2004, Blood 103: 2046-2054).

It is also important to perform the assays described above on control samples. T cell reactivity observed in patients in remission after alloSCT but not in the corresponding stem cell donor, normal individuals, or non-transplant leukemia patients can provide evidence that the candidate antigen is actually a leukemia-rejection antigen (i.e. graft vs. leukemia effects). For each patient, samples can be obtained at baseline, after alloSCT, and also from the patient's donor. A roughly equal number of samples from normal volunteers can also be collected. For each immune assay, thresholds based on internal controls can be set for positivity and outcomes can be treated as binary. Comparisons can then be made between two groups of pooled data, e.g. patient reactivity before alloSCT vs. after alloSCT; or patient reactivity vs. donor reactivity for a particular epitope. If >30 samples per group are analyzed for each comparison, the power of a chi-squared test comparing positive response rates will be >80% if the true response rate in the group with a higher rate of T cell reactivity is >65%.

Experiments can also be set up to evaluate T cell immunity in patients undergoing conventional alloSCT. The disclosure herein demonstrates that although T cells reactive with a number of candidate antigens are identifiable by tetramer analysis post-alloSCT, functional responses may be blunted. Therefore, it is advantageous to determine whether aDLI augments the function of CTL specific for leukemia antigens in vivo by providing activated CD4 T cells and other factors (e.g. cell surface CD40 ligand) during reconstitution and priming.

Without wishing to be bound by any particular theory, it is believed that the mere presence of tetramer-reactive T cells in patients after alloSCT, even if not detectable at baseline or in their stem cell donors, does not necessarily reflect a target specificity responsible for graft vs. leukemia effects. Several candidate antigens are also expressed by normal tissues targeted in graft vs. host disease and so tetramer-reactive T cells may be linked to GVHD. In addition, tetramer-reactive T cells may be the consequence, rather than the cause, of leukemia remission, whereby antigens spilled by leukemia cells killed during transplantation are scavenged by antigen presenting cells which then prime CD8+ T cells in the reconstituted host. In this latter scenario, the peptides being screened may not necessarily be presented by the leukemia cells themselves. Therefore, it is advantageous to test the ability of peptide-specific or RNA-stimulated T cells to lyse either autologous leukemia cells or allogeneic cells expressing the antigen. Tumor-reactive, and not just peptide-reactive T cells are deemed to be the most informative.

### Example 7

### HoxA9, Meis-1, and Survivin as Broadly Expressed Leukemia-Associated Antigens Recognized by CD8+ T Cells

The following experiments are conducted to assess whether HoxA9, Meis-1, and survivin represent novel T cell immune targets in leukemia that are both broadly expressed and critical for oncogenesis. Results using these antigens can be compared to those using proteinase 3, which can be used as a positive control. It is invisioned that the methodology for this analysis can also be applied to other antigens validated elsewhere herein, including WT-1, PRAME, and telomerase.

These set of experiments are therefore performed to evaluate the ability of specific CD8+ T cells isolated from patients after transplantation, or generated in vitro from normal donors, to secrete IFN-gamma and mount proliferative and cytotoxic responses against targets expressing the antigen. CTL specific for HoxA9, Meis1, and survivin is generated from patients and normal donors using peptide-based in vitro stimulation methods discussed elsewhere herein. Peptide-specific polyclonal CTL can further be purified or cloned using a system of tetramer-guided, high-speed cell sorting and stimulation with artificial antigen presenting cells expressing ligands for TCR, CD28, and 41BB, as described in Vonderheide et al. (2004, Clin. Cancer Res. 10: 828-839. T cell clones and lines can be tested for specific IFN-gamma production (e.g. intracellular IFN-gamma secretion in response to peptide-loaded vs. control-loaded T2 cells) and for cytotoxicity against leukemia targets that either do or do not express the antigen and do or do not express the appropriate HLA allele. In the first instance, CTL generated using HLA-A2-binding epitopes are examined from these three antigens. Secondly, CTL generated using autologous CD40-B cells transfected with mRNA can be examined for each tumor antigen. A “plug-and-play” vector for the production of antigen encoding mRNA has been developed and piloted for survivin. GFP mRNA can be used as a negative control and influenza mRNA can be used as a positive control. Targets in the cytotoxicity experiments also include normal cells that express the antigen and MHC class I. In the case of HoxA9, Meis I, and survivin, normal bone marrow progenitors are included. If tumor-lytic CTL also lyse normal bone marrow cells or other antigen-positive normal cells in vitro, they may not be useful. However, even for self-antigens, tumor immunity does not necessarily involve autoimmunity in normal tissues that share the target. Thus, the next set of experiments designed to evaluate the ability of specific CD8+ T cells to lyse autologous leukemia cells; for example, to test whether CTL specific for candidate leukemia-associated antigens can kill wild-type autologous leukemia cells, and not just allogeneic targets, in an antigen-specific, MHC-restricted fashion.

### Example 8

### Determine the Clinical and Immunological Impact of Vaccinating Leukemia Patients

These experiments are designed to test whether the leukemia-rejection antigens discussed elsewhere herein can be used as novel immunotherapeutics. The safety and feasibility of vaccinating patients with antigen emulsified in adjuvant and delivered with GM-CSF can be assessed. Eligible patients with myeloid leukemia would, for example receive eight vaccinations with peptide or peptides emulsified in the adjuvant Montanide ISA 51 and delivered subcutaneously with GM-CSF. Intervals between vaccinations can be about 2 weeks for the first 4 injections then approximately monthly. Patients are regularly evaluated for local tolerability, adverse events, laboratory evidence of toxicity, and evidence of progression. Blood and bone marrow samples can be obtained at baseline and periodically after vaccination.

Three cohorts of patients (5 or 8 patients/cohort, depending on toxicity) would, for example, receive doses of 10 ug, 100 ug, and 1000 ug of each peptide. An optimal dose for peptide-based trials has not been defined such that a 2-log range can be used in this study. DLT is defined as (i) any grade 3 or higher hematologic or non-hematologic toxicity; (ii) any grade 2 or higher autoimmune reaction; or (iii) any grade 2 or higher allergic reaction. A dose level can be considered too toxic if two or more patients at that level experience DLT. Dose accrual follows a standard 5+3 rule based on toxicity. Additional patients can be treated at the MTD so that a total of 12 patients (including those in the original dose level cohort) can be treated at the MTD.

The next set of experiments are designed to assess the generation of peptide-specific CTL immunity as a result of vaccination. Extensive immunologic evaluation can be performed using patient blood samples obtained before, during, and after vaccination. Assays necessary for this evaluation have been established and piloted in two previous peptide vaccination trials. Briefly, these assays include: (i) tetramer analysis, (ii) intracellular IFN-gamma analysis, and (iii) T cell cytotoxicity assays, for which methods have been previously published (Vonderheide et al., 2004, Clin. Cancer Res. 10: 828-839). Statistical considerations for the evaluation of immune-based endpoints have been calculated based on either tetramer or intracellular IFN-gamma analysis as quantitative measurements of specific T cell precursors before and after vaccination.

In the event that the vaccine strategy discussed above proves to be insufficient for the induction of CTL immunity, subsequent studies can be performed to increase the amplitude of CTL responses by employing other delivery modalities, such as peptide-loaded DCs or peptide-loaded CD40-activated B cells. Another possibility is adoptive T cell therapy, likely in combination with aDLI. A system for ex vivo CD8+ T cell expansion can also be exploited in this strategy (Maus et al., 2002, Nat. Biotechnol 20:143-148).

Immunogenicity may also be improved by incorporating additional peptides from leukemia-associated antigens, particularly epitopes restricted to MHC class II. One approach would be to use full-length mRNA to broaden the range of both CD8 and CD4 hTERT epitopes in the vaccine. mRNA-loaded DCs have been shown in vivo to trigger antigen-specific CD8 and CD4 T cells, with promising clinical activity. It is also believed that successful approaches for cancer vaccination may likely involve disruption of negative regulatory elements of both host and tumor.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A composition comprising an isolated nucleic acid encoding an epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

2. The composition of claim 1, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

3. The composition of claim 1, wherein said epitope of the antigen Meisl is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

4. A composition comprising an isolated epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

5. The composition of claim 4, wherein said epitope of the antigen HOX-A9 is selected from the group consisting of HoxKEF, HoxNLT, HoxTLD, HoxYLT, HoxRLL and HoxLLG, corresponding to the amino acid sequence set forth in SEQ ID NO:1, 2, 3, 4, 5 and 6, respectively.

6. The composition of claim 1, wherein said epitope of the antigen Meis 1 is selected from the group consisting of MeiILQ, MeiNLM, MeiPLF, MeiVLR, MeiAIY, MeiLLE, corresponding to the amino acid sequence set forth in SEQ ID NO: 7, 8, 9, 10, 11 and 12, respectively.

7. A vector comprising an isolated nucleic acid encoding an epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

8. The vector of claim 7, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

9. The vector of claim 7, wherein said epitope of the antigen Meis I is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

10. A cell comprising an isolated nucleic acid encoding an epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

11. The cell of claim 10, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

12. The cell of claim 10, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

13. The cell of claim 10, wherein said cell is a human cell.

14. A cell comprising an isolated epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

15. The cell of claim 14, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

16. The cell of claim 14, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

17. The cell of claim 14, wherein said cell is a human cell.

18. An isolated antigen-specific T cell generated according to the method comprising:
a) providing a composition comprising a peptide/MHC tetramer, wherein said peptide/MHC tetramer comprises at least one epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen;
b) contacting a population of immune cells with said composition comprising a peptide/MHC tetramer under conditions suitable for formation of a tetramer-T cell complex; and
c) substantially separating said tetramer-T cell complex from said population of immune cells;
thereby isolating said antigen-specific T cell.

19. The isolated antigen-specific T cell of claim 18, wherein said cell specifically binds to an epitope of the antigen HOX-A9, further wherein said epitope is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

20. The isolated antigen-specific T cell of claim 18, wherein said cell specifically binds to an epitope of the antigen Meis 1, further wherein said epitope is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

21. The cell of claim 18, wherein said antigen-specific T cell is a human cell.

22. An isolated antigen-specific T cell, wherein said T cell specifically binds to an epitope of the antigen HOX-A9, further wherein said epitope is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

23. The cell of claim 22, wherein said cell is a human cell.

24. An isolated antigen-specific T cell, wherein said T cell specifically binds to an epitope of the antigen Meis1, further wherein said epitope is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

25. The cell of claim 24, wherein said cell is a human cell.

26. A method of isolating an antigen-specific T cell from a population of immune cells, the method comprising:
a) providing a composition comprising a peptide/MHC tetramer, wherein said peptide/MHC tetramer comprises at least one epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen;
b) contacting said population of immune cells with said composition comprising said peptide/MHC tetramer under conditions suitable for formation of a tetramer-T cell complex; and
c) substantially separating said tetramer-T cell complex from said population of immune cells;
thereby isolating said antigen-specific T cell.

27. The method of claim 26, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

28. The method of claim 26, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

29. The method of claim 26, wherein said antigen-specific T cell is a human cell.

30. The method of claim 26, wherein said peptide/MHC tetramer is a monomer conjugated to a physical support.

31. The method of claim 30, wherein said physical support is selected from the group consisting of a microbead, a magnetic bead, a panning surface, a dense particle for density centrifugation, an adsorption column and an adsorption membrane.

32. The method of claim 30, wherein said physical support is selected from the group consisting of a streptavidin bead and a biotinavidin bead.

33. The method of claim 26, wherein said tetramer-T cell complex is substantially separated from said population of immune cells using a method selected from the group consisting of fluorescence activated cell sorting (FACS) and magnetic activated cell sorting (MACS).

34. The method of claim 26, wherein said peptide/MHC tetamer is chemically attached to the surface of said T cell.

35. The method of claim 26, wherein said population of immune cells are derived from a source selected from the group consisting of a leukapheresis product, peripheral blood, lymph node, tonsil, thymus, tissue biopsy, tumor, spleen, bone marrow, cord blood, CD34+ cells, monocytes and adherent cells.

36. A method of enriching an antigen-specific T cells from a population of immune cells, the method comprising:
a) providing a composition comprising a T cell receptor specific for at least one epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen;
b) contacting said population of immune cells with said composition comprising said peptide/MHC tetramer under conditions suitable for formation of a tetramer-T cell complex; and
c) substantially separating said tetramer-T cell complex from said population of immune cells;
thereby enriching for said antigen-specific T cell.

37. The method of claim 36, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

38. The method of claim 36, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

39. The method of claim 36, wherein said antigen-specific T cell is a human cell.

40. A method of stimulating an immune response in a mammal comprising, administering to the mammal a composition comprising an isolated nucleic acid encoding an epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

41. The method of claim 40, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

42. The method of claim 40, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

43. The method of claim 40, wherein said mammal is a human.

44. A method of stimulating an immune response in a mammal comprising, administering to the mammal a composition comprising an isolated epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

45. The method of claim 44, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO: 1, 2, 3, 4, 5 and 6, respectively.

46. The method of claim 44, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

47. The method of claim 44, wherein said mammal is a human.

48. A method of stimulating an immune response in a mammal comprising, administering to the mammal a composition comprising a cell, wherein said cell comprises an isolated nucleic acid encoding an epitope of an antigen, wherein said antigen is selected from the group consisting of proteinase 3, PRAME, HOX-A9, Meis1, WTI, survivin, telomerase, MAGE 3, and NY-ESO-1, further wherein said epitope comprises an amino acid sequence that is at least one amino acid less than the full length amino acid sequence of the antigen.

49. The method of claim 48, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

50. The method of claim 48, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

51. The method of claim 48, wherein said mammal is a human.

52. The method of claim 48, wherein said cell is a human cell.

53. A method of stimulating an immune response in a mammal comprising, administering to the mammal a composition comprising a cell, wherein said cell comprises an epitope of an antigen, wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

54. The method of claim 53, wherein said epitope of the antigen HOX-A9 is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

55. The method of claim 53, wherein said epitope of the antigen Meis1 is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

56. The method of claim 53, wherein said mammal is a human.

57. The method of claim 53, wherein said cell is a human cell.

58. A method for treating cancer in a mammal, the method comprising administering a composition to said mammal, wherein said composition comprises a peptide/MHC tetramer comprising at least one epitope of an antigen, further wherein said antigen is selected from the group consisting of proteinase 3, PRAME, HOX-A9, Meis1, WT1, survivin, telomerase, MAGE 3, and NY-ESO-1, further wherein said epitope comprises an amino acid sequence that is at least one amino acid less than the full length amino acid sequence of the antigen.

59. The method of claim 58, wherein said epitope is selected from the group consisting of HoxKEF, HoxNLT, HoxTLD, HoxYLT, HoxRLL and HoxLLG, corresponding to the amino acid sequences set forth in SEQ ID NO:1, 2, 3, 4, 5 and 6, respectively.

60. The method of claim 58, wherein said epitope is selected from the group consisting of MeiILQ, MeiNLM, MeiPLF, MeiVLR, MeiAIY, MeiLLE, corresponding to the amino acid sequences set forth in SEQ ID NO: 7, 8, 9, 10, 11 and 12, respectively.

61. The method of claim 58, wherein said cancer selected from the group consisting of melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, pancreatic cancer, esophageal cancer, brain cancer, lung cancer, ovarian cancer, cervical cancer, multiple myeloma, hepatoma, acute lymphoblastic leukemia (ALL), acute myelogenous leukemia (AML), chronic myelogenous leukemia (CML), and chronic lymphocytic leukemia (CLL).

62. The method of claim 58, wherein said mammal is a human.


SEQUENCE LISTING (17 sequences)

SEQ ID NO: 1 - protein, 9 aa, Artificial
  human-derived peptide
KEFLFNMYL

SEQ ID NO: 2 - protein, 9 aa, Artificial
  human-derived peptide
NLTERQVKI

SEQ ID NO: 3 - protein, 9 aa, Artificial
  human-derived peptide
TLDTHTLSL

SEQ ID NO: 4 - protein, 10 aa, Artificial
  human-derived peptide
YLTRDRRYEV

SEQ ID NO: 5 - protein, 10 aa, Artificial
  human-derived peptide
RLLNLTERQV

SEQ ID NO: 6 - protein, 10 aa, Artificial
  human-derived peptide
LLGADAADEL

SEQ ID NO: 7 - protein, 9 aa, Artificial
  human-derived peptide
ILQVNNWFI

SEQ ID NO: 8 - protein, 8 aa, Artificial
  human-derived peptide
NLMIQAIQ

SEQ ID NO: 9 - protein, 9 aa, Artificial
  human-derived peptide
PLFPLLALV

SEQ ID NO: 10 - protein, 9 aa, Artificial
  human-derived peptide
VLRFHLLEL

SEQ ID NO: 11 - protein, 10 aa, Artificial
  human-derived peptide
AIYGHPLFPL

SEQ ID NO: 12 - protein, 10 aa, Artificial
  human-derived peptide
LLELEKVHEL

SEQ ID NO: 13 - protein, 9 aa, Artificial
  human-derived peptide
FLWGPRALV

SEQ ID NO: 14 - protein, 272 aa, Homo sapiens
MATTGALGNYYVDSFLLGADAADELSVGRYAPGTLGQPPRQAATLAEHPDFSPCSFQSKATVFGASWNPVHAAGANAVPAAVYHHHHHHPYVHPQAPVAAAAPDGRYMRSWLEPTPGALSFAGLPSSRPYGIKPEPLSARRGDCPTLDTHTLSLTDYACGSPPVDREKQPSEGAFSENNAENESGGDKPPIDPNNPAANWLHARSTRKKRCPYTKHQTLELEKEFLFNMYLTRDRRYEVARLLNLTERQVKIWFQNRRMKMKKINKDRAKDE

SEQ ID NO: 15 - DNA, 2076 bp, Homo sapiens
agttgttacatgaaatctgcagtttcataatttccgtgggtcgggccgggcgggccaggcgctgggcacggtgatggccaccactggggccctgggcaactactacgtggactcgttcctgctgggcgccgacgccgcggatgagctgagcgttggccgctatgcgccggggaccctgggccagcctccccggcaggcggcgacgctggccgagcaccccgacttcagcccgtgcagcttccagtccaaggcgacggtgtttggcgcctcgtggaacccagtgcacgcggcgggcgccaacgctgtacccgctgcggtgtaccaccaccatcaccaccacccctacgtgcacccccaggcgcccgtggcggcggcggcgccggacggcaggtacatgcgctcctggctggagcccacgcccggtgcgctctccttcgcgggcttgccctccagccggccttatggcattaaacctgaaccgctgtcggccagaaggggtgactgtcccacgcttgacactcacactttgtccctgactgactatgcttgtggttctcctccagttgatagagaaaaacaacccagcgaaggcgccttctctgaaaacaatgctgagaatgagagcggcggagacaagccccccatcgatcccaataacccagcagccaactggcttcatgcgcgctccactcggaaaaagcggtgcccctatacaaaacaccagaccctggaactggagaaagagtttctgttcaacatgtacctcaccagggaccgcaggtacgaggtggctcgactgctcaacctcaccgagaggcaggtcaagatctggttccagaaccgcaggatgaaaatgaagaaaatcaacaaagaccgagcaaaagacgagtgatgccatttgggcttatttagaaaaaagggtaagctagagagaaaaagaaagaactgtccgtcccccttccgccttctcccttttctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgccaaataaaatatattgattcttttctaaaaaaaaaaaaaaaaaaaa

SEQ ID NO: 16 - protein, 390 aa, Homo sapiens
MAQRYDDLPHYGGMDGVGIPSTMYGDPHAARSMQPVHHLNHGPPLHSHQYPHTAHTNAMAPSMGSSVNDALKRDKDAIYGHPLFPLLALIFEKCELATCTPREPGVAGGDVCSSESFNEDIAVFAKQIRAEKPLFSSNPELDNLMIQAIQVLRFHLLELEKVHELCDNFCHRYISCLKGKMPIDLVIDDREGGSKSDSEDITRSANLTDQPSWNRDHDDTASTRSGGTPGPSSGGHTSHSGDNSSEQGDGLDNSVASPSTGDDDDPDKDKKRHKKRGIFPKVATNIMRAWLFQHLTHPYPSEEQKKQLAQDTGLTILQVNNWFINARRRIVQPMIDQSNRAVSQGTPYNPDGQPMGGFVMDGQQHMGIRAPGPMSGMGMNMGMEGQWHYM

SEQ ID NO: 17 - DNA, 2902 bp, Homo sapiens
atttgaggtgttctgaccagaagaagacagagcggatgatcattcattcaccacgttgacaacctcgcctgtgattgacagctggagtggcagaaagccatgagatttggtagttgggtctgaggggcgctcttttttttccttttctttctttctttcttttttttttttaaactgatttttgggggagagaagatctgcttttttttgcccccgctgctgtcttggaaacggagcgcttttatgctcagtgactcgggcgctttgcttcaggtcccgtagaccgaagatctgggaccagtagctcacgttgctggagacgttaagggatttttcgtcgtgcttttttttttttttttttttccgggggagtttgaatatttgtttcttttcacactggccttaaagaggatatattagaagttgaagtaggaagggagccagagaggccgatggcgcaaaggtacgacgatctaccccattacgggggcatggatggagtaggcatcccctccacgatgtatggggacccgcatgcagccaggtccatgcagccggtccaccacctgaaccacgggcctcctctgcactcgcatcagtacccgcacacagctcataccaacgccatggcccccagcatgggctcctctgtcaatgacgctttaaagagagataaagatgccatttatggacaccccctcttccctctcttagcactgatttttgagaaatgtgaattagctacttgtaccccccgcgagccgggggtggcgggcggggacgtctgctcgtcagagtcattcaatgaagatatagccgtgttcgccaaacagattcgcgcagaaaaacctctattttcttctaatccagaactggataacttgatgattcaagccatacaagtattaaggtttcatctattggaattagagaaggtacacgaattatgtgacaatttctgccaccggtatattagctgtttgaaagggaaaatgcctatcgatttggtgatagacgatagagaaggaggatcaaaatcagacagtgaagatataacaagatcagcaaatctaactgaccagccctcttggaacagagatcatgatgacacggcatctactcgttcaggaggaaccccaggcccttccagcggtggccacacgtcacacagtggggacaacagcagtgagcaaggtgatggcttggacaacagtgtagcttcccccagcacaggtgacgatgatgaccctgataaggacaaaaagcgtcacaaaaagcgtggcatctttcccaaagtagccacaaatatcatgagggcgtggctgttccagcatctaacacacccttacccttctgaagaacagaaaaagcagttggcacaagacacgggactcaccatccttcaagtgaacaattggtttattaatgcccggagaagaatagtgcagcccatgatagaccagtccaaccgagcagtaagtcaaggaacaccttataatcctgatggacagcccatgggaggtttcgtaatggacggtcagcaacatatgggaattagagcaccaggacctatgagtggaatgggcatgaatatgggcatggaggggcagtggcactacatgtaaccttcatctagttaaccaatcgcaaagcaagggggaaaatttcttacagggctgcaaagtatgccaggggagtatgtagcccggggtggtccaatgggtgtgagtatgggacagccaagttatacccaaccccagatgcccccccatcctgctcagctgcgtcatgggccccccatgcatacgtacattcctggacaccctcaccacccaacagtgatgatgcatggaggaccgccccaccctggaatgccaatgtcagcatcaagccccacagttcttaatacaggagacccaacaatgagtggacaagtcatggacattcatgctcagtagcttaagggaatatgcattgtctgcaatggtgactgatttcaaatcatgttttttctgcaatgactgtggagttccattcttggcatctactctggaccaaggagcatccctaattcttcatagggacctttaaaaagcaggaaataccaactgaagtcaatttgggggacatgctaaataactatataagacattaagagaacaaagagtgaaatattgtaaatgctattatactgttatccatattacgttgtttcttatagattttttaaaaaaaatgtgaaatttttccacactatgtgtgttgtttccatagctcttcacttcctccagaagcctccttacattaaaaagccttacagttatcctgcaagggacaggaaggtctgatttgcaggatttttagagcattaaaataactatcaggcagaagaatctttcttctcgcctaggatttcagccatgcgcgcgctctctctctttctctctcttttcctctctctccctctttctagcctggggcttgaatttgcatgtctaattcatttactcaccatatttgaattggcctgaacagatgtaaatcgggaaggatgggaaaaactgcagtcatcaacaatgattaatcagctgttgcaggcagtgtcttaaggagactggtaggaggaggcatggaaaccaaaaggccgtgtgtttagaagcctaattgtcacatcaagcatcattgtccccatgcaacaaccaccaccttatacatcacttcctgttttaagcagctctaaaacatagactgaagatttatttttaatatgttgactttatttctgagcaaagcatcggtcatgtgtgtattttttcatagtcccaccttggagcatttatgtagacattgtaaataaattttgtgcaaaaaggactggaaaaatgaaaaaaaaaaaaaaaaa